(19) United States
(12) Patent Application Publication
(10) Pub. No.: US 2024/0167063 A1
(43) Pub. Date: May 23, 2024

(54) RECOMBINANT HOST CELLS AND METHODS FOR THE PRODUCTION OF ISOBUTYRIC ACID

(71) Applicant: LYGOS, INC., Berkeley, CA (US)

(72) Inventors: Jeffrey DIETRICH, Berkeley, CA (US); Mario OUELLET, Berkeley, CA (US); Andrew Jonathan CONLEY, Berkeley, CA (US)

(73) Assignee: LYGOS, INC., Berkeley, CA (US)

(21) Appl. No.: 18/144,378

(22) Filed: May 8, 2023

Related U.S. Application Data
(63) Continuation of application No. 16/961,057, filed on Jul. 9, 2020, now Pat. No. 11,680,280.
(60) Provisional application No. 62/615,202, filed on Jan. 9, 2018.

(51) Int. Cl. C12P 7/52

(52) U.S. Cl. CPC C12P 7/52; C12Y 101/01086; C12Y 102/01005; C12Y 202/01006; C12Y 401/01072; C12Y 402/01009

(57) ABSTRACT

Methods and materials related to producing isobutyric acid are disclosed. Specifically, isolated nucleic acids, polypeptides, host cells, methods and materials for producing isobutyric by direct microbial fermentation from a carbon source are disclosed.

### CROSS-REFERENCE TO RELATED APPLICATIONS

This application claims the benefit of priority under 35 U.S.C. 119(e) and Article 2 of the Paris Convention for the Protection of Industrial Property (1883) to U.S. provisional application Ser. No. 62/615,202 filed 9 Jan. 2018, the entire contents of which are incorporated herein by this reference.

### REFERENCE TO SEQUENCE LISTING

This application contains a Sequence Listing submitted via EFS-web and is hereby incorporated by reference in its entirety. The ASCII copy, created on Jan. 4, 2019, is named LYGOS_0011_01_WO_ST25 and is 101 KB in size.

### FIELD

Embodiments herein relate to recombinant host cells and methods for producing isobutyric acid in recombinant host cells. In certain embodiments, the methods include using recombinant host cells and methods to produce isobutyric acid by microbial fermentation using a carbon source, such as a sugar feedstock. In some embodiments, the recombinant host cells include archaeal, prokaryotic, or eukaryotic cells. In some embodiments, the recombinant host cells contain heterologous nucleic acids encoding one or more isobutyric acid pathway enzyme, wherein the pathway enzymes may include acetolactate synthase, ketol-acid reductoisomerase, dihydroxy-acid dehydratase, branched-chain-2-oxoacid decarboxylase and isobutyraldehyde dehydrogenase.

### BACKGROUND

The long-term economic and environmental concerns associated with the petrochemical industry have provided the impetus for increased research, development, and commercialization of processes for conversion of carbon feedstocks into chemicals that can replace those petroleum feedstocks. One approach is the development of biorefining processes to convert renewable feedstocks into products that can replace petroleum-derived chemicals. Two common goals in improving a biorefining process include achieving a lower cost of production and reducing detrimental effects on the environment.

Isobutyric acid (CAS No. 79-31-2) is an important intermediate chemical that can be dehydrogenated to produce the industrially important compound methacrylic acid (see Ullmann's Encyclopedia of Industrial Chemistry), a key component in the manufacture of polymer dispersions (coatings, paints and printing inks), adhesives, binding agents, super absorbent polymers, flocculants, detergents, varnishes, fibers and plastics, as well as chemical intermediates. Esters of isobutyric acid (for example, methyl butyrate and ethyl butyrate) are also important solvents, coalescents, extractants, flavor and fragrance compounds, and diluents used in paints, lacquers, and coatings.

Isobutyric acid can be produced by several routes of chemical synthesis, each requiring a petrochemical feedstock. Isobutyric acid may be prepared by: 1) carbonylation of propene with a strong acid catalyst; 2) hydrolysis of isobutyronitrile with alkalis; 3) oxidation of isobutanol with potassium dichromate and sulfuric acid; or 4) reacting sodium amalgam with methacrylic acid.

The present disclosure provides recombinant host cells and methods to produce isobutyric acid by microbial fermentation using a carbon source, such as a sugar feedstock. The methods described herein not only comprise a renewable and cheaper starting material compared to petrochemicals, but also contribute to energy independence. The methods described herein enable higher fermentation yields and productivities in the production of isobutyric acid.

### SUMMARY

In a first aspect, one embodiment of this disclosure provides recombinant host cells capable of producing isobutyric acid containing one or more heterologous nucleic acids that encode the isobutyric acid biosynthetic pathway, wherein the pathway enzymes include acetolactate synthase, ketol-acid reductoisomerase, dihydroxy-acid dehydratase, branched-chain-2-oxoacid decarboxylase and isobutyraldehyde dehydrogenase.

In some embodiments, the recombinant host cell is a yeast cell. In certain embodiments, the yeast cell belongs to the Issatchenkia orientalis/Pichia fermentans clade. In some embodiments, the yeast cell belongs to the genus Pichia, Issatchenkia or Candida. In some embodiments, the yeast cell is Pichia kudriavzevii. In some embodiments, the yeast cell belongs to the Saccharomyces clade. In some embodiments, the yeast cell is Saccharomyces cerevisiae.

In other embodiments, the recombinant host cell is a prokaryotic cell. In some embodiments, the prokaryotic cell belongs to the genus Escherichia, Corynebacterium, Bacillus, or Lactococcus. In some embodiments, the prokaryotic cell is Escherichia coli, Corynebacterium glutamicum, Bacillus subtilis, or Lactococcus lactis.

In some embodiments, the recombinant host cells contain heterologous nucleic acids encoding an acetolactate synthase with substantial amino acid sequence homology to SEQ ID NO: 1 or SEQ ID NO: 19. In some embodiments, the recombinant host cells contain heterologous nucleic acids encoding a ketol-acid reductoisomerase with substantial homology to the amino acid sequence represented by SEQ ID NO: 2 or SEQ ID NO: 21. In some embodiments, the recombinant host cells contain heterologous nucleic acids encoding a dihydroxy-acid dehydratase with substantial homology to the amino acid sequence represented by SEQ ID NO: 3 or SEQ ID NO: 22. In some embodiments, the recombinant host cells contain heterologous nucleic acids encoding a branched-chain-2-oxoacid decarboxylase with substantial homology to the amino acid sequence represented by SEQ ID NO: 4 or SEQ ID NO: 23. In some embodiments, the recombinant host cells contain heterologous nucleic acids encoding a isobutyraldehyde dehydrogenase with substantial homology to the amino acid sequence represented by SEQ ID NO: 5 or SEQ ID NO: 24.

In another aspect, some embodiments of this disclosure provide recombinant host cells that further contain one or more heterologous nucleic acids encoding one or more ancillary proteins that function in redox cofactor recycling, redox cofactor biogenesis, or organic acid transport. In some embodiments, the one or more ancillary proteins include mitochondrial external NADH dehydrogenase, water-forming NADH oxidase, isobutyric acid transporter, or combination thereof. Both the mitochondrial external NADH dehydrogenase and water-forming NADH oxidase oxidize NADH to NAD+, recycling the cofactor necessary for efficient isobutyric acid pathway activity. In one embodiment, the ancillary protein is an external NADH dehydrogenase, NdeI, derived from P. kudriavzevii. In some embodiments, the ancillary protein has at least 60%, at least 70%, at least 80%, at least 90%, or at least 95% amino acid identity to the amino acid sequence represented by SEQ ID NO: 6. In one embodiment, the ancillary protein is a water forming NADH oxidase derived from Lactococcus lactis (NoxE; UniProt ID: A2RIB7; SEQ ID NO: 7). In some embodiments, the ancillary protein has at least 60%, at least 70%, at least 80%, at least 90%, or at least 95% amino acid identity to the amino acid sequence represented by SEQ ID NO: 7. In one embodiment, the ancillary protein has isobutyric acid transporter activity. Examples of isobutyric acid transporters include Saccharomyces cerevisiae PDR12, Saccharomyces cerevisiae WAR1, and Kluyveromyces marxianus PDC12 and in some embodiments the ancillary protein has at least 60%, at least 70%, at least 80%, at least 90%, or at least 95% amino acid identity to S. cerevisiae PDR12, S. cerevisiae WAR1, and/or K. marxianus PDC12.

In another aspect, some embodiments of this disclosure provide recombinant host cells that further include a genetic disruption of one or more genes wherein the one or more genes encodes pyruvate decarboxylase, pyruvate dehydrogenase, alcohol dehydrogenase, acetaldehyde dehydrogenase, or glycerol-3-phosphate dehydrogenase, or combination thereof. In some embodiments, the one or more genes encodes an amino acid sequence that has at least 60%, at least 70%, at least 80%, at least 90%, or at least 95% homology to SEQ ID NO: 12, encoding P. kudriavzevii NAD-dependent glycerol-3-phosphate dehydrogenase (PkGPD1). Glycerol-3-phosphate dehydrogenase catalyzes the conversion of dihydroxyacetone phosphate to glycerol 3-phosphate and its activity can lead to production of the byproduct glycerol. As such, deletion or disruption of the nucleic acid(s) encoding glycerol-3-phosphate dehydrogenase decreases glycerol production as compared to a control, parental host cell that does not comprise said deletion or disruption. In some embodiments, the recombinant host cell comprising deletion or disruption of one or more nucleic acids encoding one or more glycerol-3-phosphate dehydrogenase enzymes produces less than 2.5 g/l glycerol. In some embodiments, the one or more genes encodes a pyruvate decarboxylase with amino acid sequence represented by SEQ ID NO: 8, SEQ ID NO: 9, SEQ ID NO: 10, or a combination thereof. Pyruvate decarboxylase catalyzes the conversion of pyruvate to acetaldehyde and its activity can lead to production of the byproduct ethanol. As such, deletion or disruption of the nucleic acid(s) encoding one or more pyruvate decarboxylase enzymes decreases ethanol production as compared to a control, parental host cell that does not comprise said deletion or disruption. In some embodiments, the recombinant host cell comprising deletion or disruption of one or more nucleic acids encoding one or more pyruvate decarboxylase enzymes produces less than 2.5 g/l ethanol. In some embodiments, the one or more genes encodes an alcohol dehydrogenase with amino acid sequence represented by SEQ ID NO: 13, SEQ ID NO: 14, or a combination thereof. Alcohol dehydrogenase catalyzes the conversion of aldehydes to alcohols, and as such can convert isobutyraldehyde to isobutanol, an undesirable byproduct. Deletion or disruption of the nucleic acid(s) encoding one or more alcohol dehydrogenase enzymes decreases ethanol production as compared to a control, parental host cell that does not comprise said deletion or disruption. In some embodiments, the recombinant host cell comprising deletion or disruption of one or more nucleic acids encoding one or more alcohol dehydrogenase enzymes produces less than 5 g/l isobutanol. In some embodiments, the one or more genes encodes an acetaldehyde dehydrogenase with amino acid sequence represented by SEQ ID NO: 15, SEQ ID NO: 16, SEQ ID NO: 17, SEQ ID NO: 18, or a combination thereof. Acetaldehyde dehydrogenase catalyzes the conversion of acetaldehyde to acetate, an undesirable byproduct. Deletion or disruption of the nucleic acid(s) encoding one or more acetaldehyde dehydrogenase enzymes decreases acetate production as compared to a control, parental host cell that does not comprise said deletion or disruption. In some embodiments, the recombinant host cell comprising deletion or disruption of one or more nucleic acids encoding one or more acetaldehyde dehydrogenase enzymes produces less than 5 g/l acetate.

In another aspect, some embodiments of this disclosure provide a method for the production of isobutyric acid that includes culturing the recombinant host cells detailed in this disclosure for a sufficient period of time to produce isobutyric acid. In some embodiments, the method further includes an oxygen transfer rate greater than 10 mmol/l/hr. In some embodiments, the method further includes an operational temperature of between about 25° C. and about 45° C. In some embodiments, the method further includes a final fermentation broth pH of about pH 5. In some embodiments, the method produces a solution containing at least 50 g/l isobutyric acid. In some embodiments, the method further includes providing at least 100 g-glucose to the recombinant host cell and producing an isobutyric acid yield of at least 25%.

### BRIEF DESCRIPTION OF THE DRAWINGS

The following drawing forms part of the instant specification and is included to further demonstrate certain aspects of particular embodiments herein. Those skilled in the art will understand that the drawing described herein is for illustration purposes only. The drawing is not intended to limit the scope of the present disclosure.

FIG. 1. represents an example of an isobutyric acid pathway, which may be used to convert two molecules of pyruvate into one molecule isobutyric acid.

### DETAILED DESCRIPTION

The present disclosure provides recombinant host cells and materials and methods for the biological production and purification of isobutyric acid using recombinant host cells.

While the present disclosure describes aspects and specific embodiments, those skilled in the art will recognize that various changes may be made and equivalents may be substituted without departing from embodiments of the present disclosure. The present disclosure is not limited to particular nucleic acids, expression vectors, enzymes, biosynthetic pathways, host microorganisms, or processes, as such may vary. The terminology used herein is for the purposes of describing particular aspects and embodiments only and is not to be construed as limiting. In addition, many modifications may be made to adapt a particular situation, material, composition of matter, process, process steps or process flows, in accordance with embodiments of the present disclosure. All such modifications are within the scope of the claims appended hereto.

### Definitions

Unless defined otherwise, all technical and scientific terms used herein have the same meaning as is commonly understood by one of ordinary skill in the art to which this disclosure pertains.

The term “accession number” and similar terms such as “protein accession number”, “UniProt ID”, “gene ID” and “gene accession number” refer to designations given to specific proteins or genes. These identifiers described a gene or protein sequence in publicly accessible databases such as the National Center for Biotechnology Information (NCBI).

A plus (+) in a consensus sequence indicates any amino acid may be present at the specified position. Thus, a plus in a consensus sequence herein indicates a position at which the amino acid is generally non-conserved; a homologous protein sequence, when aligned with the consensus sequence, can have any amino acid at the indicated “+” position.

As used herein, when one of multiple closely related amino acids is found at the aligned position of an amino acid sequence, the following one-letter symbol is used—“B” refers to aspartic acid or asparagine; “Z” refers to glutamine or glutamic acid; “J” refers to leucine or isoleucine; and “X” or “+” refers to any amino acid. Generally, homologous proteins share substantial sequence identity. Sets of homologous proteins generally possess one or more specific amino acids that are conserved across all members of the consensus sequence protein class. The percent sequence identity of a protein relative to a consensus sequence is determined by aligning the protein sequence against the consensus sequence. Practitioners in the art will recognized that various sequence alignment algorithms are suitable for aligning a protein with a consensus sequence. See, for example, Needleman, SB, et al., “A general method applicable to the search for similarities in the amino acid sequence of two proteins.” Journal of Molecular Biology 48 (3):443-53 (1970). Following alignment of the protein sequence relative to the consensus sequence, the percentage of positions where the protein possesses an amino acid described by the same position in the consensus sequence determines the percent sequence identity. When a degenerate amino acid is present (i.e., B, Z, X, J or “+”) in a consensus sequence, any of the amino acids described by the degenerate amino acid may be present in the protein at the aligned position for the protein to be identical to the consensus sequence at the aligned position.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild-type, in the case of an endogenous enzyme. Those skilled in the art appreciate that overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “expression vector” or “vector” refer to a nucleic acid and/or a composition containing a nucleic acid that can be introduced into a host cell, e.g., by transduction, transformation, or infection, such that the cell then produces (i.e., expresses) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acids into the host cell, such as the materials associated with a virus, liposome, protein coating, or the like. Expression vectors suitable for use in various aspects and embodiments of the present disclosure include those into which a nucleic acid sequence can be, or has been, inserted, along with any preferred or required operational elements. Thus, an expression vector can be transferred into a host cell and, typically, replicated therein (although, on can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromasomally is employed. Typical expression vectors include plasmids, and expression vectors typically contain the operational elements required for transcription of a nucleic acid in the vector. Such plasmids, as well as other expression vectors, are described herein or are well known to those of ordinary skill in the art.

The terms “ferment”, “fermentative”, and “fermentation” are used herein to describe culturing microbes under conditions to produce useful chemicals, including but not limited to conditions under which microbial growth, be it aerobic or anaerobic, occurs.

The term “heterologous” as used herein refers to a material that is non-native to a particular cell. For example, a nucleic acid is heterologous to a cell, and so is a “heterologous nucleic acid” with respect to that cell, if at least one of the following is true: 1) the nucleic acid is not naturally found in that cell (that is, it is an “exogenous” nucleic acid); 2) the nucleic acid is naturally found in a given host cell (that is, “endogenous to”), but the nucleic acid or the RNA or protein resulting from transcription and translation of this nucleic acid is produced or present in the host cell in an unnatural (e.g., greater or lesser than naturally present) amount; 3) the nucleic acid contains a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (e.g., higher or lower or different) activity; and/or 4) the nucleic acid includes two or more nucleotide sequences that are not found in the same relationship to each other in the cell. As another example, a protein is heterologous to a host cell if it is produced by translation of RNA or the corresponding RNA is produced by transcription of a heterologous nucleic acid. Further, a protein is also heterologous to a host cell if it is a mutated version of an endogenous protein, and the mutation was introduced by genetic engineering.

The term “homologous”, as well as variations thereof, such as “homology”, refers to the similarity of a nucleic acid or amino acid sequence, typically in the context of a coding sequence for a gene or the amino acid sequence of a protein. Homology searches can be employed using a known amino acid or coding sequence (the “reference sequence”) for a useful protein to identify homologous coding sequences or proteins that have similar sequences and thus are likely to perform the same useful function as the protein defined by the reference sequence. As will be appreciated by those of skill in the art, a protein having homology to a reference protein is determined, for example and without limitation, by a BLAST (https://blast.ncbi.nlm.nih.gov) search. A protein with high percent homology is highly likely to carry out the identical biochemical reaction as the reference protein. In some cases, two enzymes having greater than 50% identity will carry out identical biochemical reactions, and the higher the identity, i.e., 50%, 60%, 70%, 80%, 90% or greater than 95% identity, the more likely the two proteins have the same or similar function. A protein with at least 60% homology, and in some cases, at least 45% homology, to its reference protein is defined as substantially homologous. Proteins that share a specific function are not always defined or limited by percent sequence identity. In some cases, a protein with low percent sequence identity to a reference protein is able to carry out the identical biochemical reaction as the reference protein. Such proteins may share three-dimensional structure which enables shared specific functionality, but not necessarily sequence similarity. Such proteins may share an insufficient amount of sequence similarity to indicate that they are homologous via evolution from a common ancestor and would not be identified by a BLAST search or other sequence-based searches. Thus, in some embodiments of the present disclosure, homologous proteins comprise proteins that lack substantial sequence similarity but share substantial functional similarity and/or substantial structural similarity.

The terms “host cell”, “host microorganism” and “host microbe” are used interchangeably herein to refer to a living cell that can be (or has been) transformed via insertion of an expression vector. A host cell or microorganism as described herein may be a prokaryotic cell (e.g., a microorganism of the kingdom Eubacteria) or a eukaryotic cell. As will be appreciated by one of skill in the art, a prokaryotic cell lacks a membrane-bound nucleus, while a eukaryotic cell has a membrane-bound nucleus.

The terms “isolated” or “pure” refer to material that is substantially, e.g., greater than 50% or greater than 75%, or essentially, e.g., greater than 90%, 95%, 98% or 99%, free of components that normally accompany it in its native state, e.g., the state in which it is naturally found or the state in which it exists when it is first produced. Additionally, any reference to a “purified” material is intended to refer to an isolated or pure material.

As used herein, the term “nucleic acid” and variations thereof shall be generic to polydeoxyribonucleotides (containing 2-deoxy-D-ribose), polyribonucleotides (containing D-ribose), segments of polydeoxyribonucleotides, and segments of polyribonucleotides. “Nucleic acid” can also refer to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, e.g., as in the sequence of a coding region, the nucleic acid in a gene composed of a promoter and coding region, which encodes the product of a gene, which may be an RNA, e.g., a rRNA, tRNA, or mRNA, or a protein (where a gene encodes a protein, both the mRNA and the protein are “gene products” of that gene).

In the present disclosure, the term “genetic disruption” refers to several ways of altering genomic, chromosomal or plasmid-based gene expression. Genetic disruptions encompass nucleic acid deletions, nucleic acid insertions, nucleic acid substitutions, nucleic acid mutations, knockouts, premature stop codons and transcriptional promoter modifications. In the present disclosure, “genetic disruption” is used interchangeably with “genetic modification”, “genetic mutation” and “genetic alteration.” Genetic disruptions give rise to altered gene expression and or altered protein activity. Altered gene expression encompasses decreased, eliminated and increased gene expression levels. In some examples, gene expression results in protein expression, in which case the term “gene expression” is synonymous with “protein expression”.

The term “operably linked” refers to a functional linkage between a nucleic acid expression control sequence (such as a promoter, ribosome-binding site, and transcription terminator) and a second nucleic acid sequence, the coding sequence or coding region, wherein the expression control sequence directs or otherwise regulates transcription and/or translation of the coding sequence.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

As used herein, the term “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

The terms “transduce”, “transform”, “transfect”, and variations thereof as used herein refers to the introduction of one or more nucleic acids into a cell. For practical purposes, the nucleic acid must be stably maintained or replicated by the cell for a sufficient period of time to enable the function(s) or product(s) it encodes to be expressed for the cell to be referred to as “transduced”, “transformed”, or “transfected”. As will be appreciated by those of skill in the art, stable maintenance or replication of a nucleic acid may take place either by incorporation of the sequence of nucleic acids into the cellular chromosomal DNA, e.g., the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, e.g., viruses, of the same type as the original transducing expression vector to other microorganisms, wherein the progeny expression vectors possess the same ability to reproduce.

As used herein, “isobutyric acid” is intended to mean the molecule having the chemical formula (CH₃)₂CHCO₂H and a molecular mass of 88.11 g/mol (CAS # 79-31-2). The terms “isobutyric acid”, “isobutanoic acid”, “2-methylpropanoic acid”, “dimethylacetic acid”, “isopropylformic acid” and “2-methylpropionic acid” are used interchangeably in the present disclosure, and practitioners skilled in the art understand that these terms are synonyms.

Herein, “isobutyrate” is also used interchangeably with “isobutyric acid”, “isobutyrate anion” and practitioners skilled in the art understand that these terms are synonyms. In conditions with pH values higher than the pKa of isobutyric acid (e.g., about pH>4.84 when using a sodium base, such as sodium hydroxide), isobutyric acid is deprotonated to the isobutyrate anion C₄H₇O₂⁻.

The term “isobutyrates” is intended to mean a variety of isobutyrate salt forms and is used interchangeably with “isobutyrate salts”. The isobutyrate anion is capable of forming an ionic bond with a cation to produce an isobutyrate salt. Non-limiting examples of isobutyrates include ammonium isobutyrate (CAS # 22228-82-6), calcium isobutyrate (CAS # 533-09-4), potassium isobutyrate (CAS # 19455-20-0), and sodium isobutyrate (CAS # 996-30-5).

As used herein, “isobutyric acid” is defined as “bio-based isobutyric acid” and “isobutyrate” is defined as “bio-based isobutyrate.” As used herein, the prefix “bio-” or the adjective “bio-based” may be used to distinguish e biologically-produced isobutyric acid and isobutyrates from those that are derived from petroleum feedstocks. The isobutyric acid and isobutyrates of the present disclosure are synthesized from biologically produced organic components by a fermenting microorganism. For example, isobutyric acid and isobutyrates are synthesized from the fermentation of a carbon source, for example sugars, by recombinant host cells of the present disclosure

The term “byproduct” or “by-product” means an undesired chemical related to the biological production of a target molecule. In the present disclosure, “byproduct” is intended to mean any amino acid, amino acid precursor, chemical, chemical precursor, organic acid, organic acid precursor, biofuel, biofuel precursor, or small molecule, that may accumulate during biosynthesis of isobutyric acid. In some cases, “byproduct” accumulation may decrease the yields, titers or productivities of the target product (e.g., isobutyric acid) in a fermentation.

The redox cofactor nicotinamide adenine dinucleotide, NAD, comes in two forms—phosphorylated and un-phosphorylated. As used herein, the term “NAD(P)” refers to either phosphorylated (NADP) and/or un-phosphorylated (NAD) forms and encompasses oxidized versions (NAD⁺ and NADP⁺) and reduced versions (NADH and NADPH) of both forms. The term “NAD(P)⁺” refers to the oxidized versions of phosphorylated and un-phosphorylated NAD, i.e., NAD⁺ and NADP⁺. Similarly, the term “NAD(P)H” refers to the reduced versions of phosphorylated and un-phosphorylated NAD, i.e., NADH and NADPH. When NAD(P)H is used to describe the redox cofactor in an enzyme catalyzed reaction, it indicates that NADH and/or NADPH is used. Similarly, when NAD(P)⁺ is the notation used, it indicates that NAD⁺ and/or NADP⁺ is used. Those skilled in the art will also appreciate that while many proteins may only bind either a phosphorylated or un-phosphorylated cofactor, there are redox cofactor promiscuous proteins, natural or engineered, that are indiscriminate; in these cases, the protein may use either NADH and/or NADPH.

Various values for temperatures, titers, yields, oxygen transfer rate (OTR), and pH are recited in the description and in the claims. It should be understood that these values are not exact. However, the values can be approximated to the rightmost/last/least significant figure, except where otherwise indicated. For example, a temperature range of from about 30° C. to about 42° C. covers the range 25° C. to 44° C. It should be understood that numerical ranges recited can also include the recited minimum value and the recited maximum value when the values are approximated to the rightmost/last/least significant figure. For example, a temperature range of from about 25ºC to about 50° C. covers the range of 25° C. to 50° C.

### Recombinant Host Cells for Production of Isobutyric Acid Host Cells

The present disclosure provides recombinant host cells engineered to produce isobutyric acid, wherein the recombinant host cells may contain one or more heterologous nucleic acids encoding one or more isobutyric acid pathway enzymes. In certain embodiments, the recombinant host cells may further contain one or more heterologous nucleic acids encoding ancillary gene products (i.e., gene products other than the isobutyric acid pathway enzymes) that improve yields, titers and/or productivities of isobutyric acid. In particular embodiments, the recombinant cells further contain genetic disruptions that improve yields, titers and/or productivities of isobutyric acid. In some embodiments, the recombinant host cells are capable of producing isobutyric acid under aerobic conditions. In some embodiments, the recombinant host cells are capable of producing isobutyric acid under substantially anaerobic conditions.

In many embodiments, the recombinant host cells contain one or more heterologous nucleic acids encoding an isobutyric acid pathway enzyme. The recombinant host cells produce isobutyric acid at increased titers, yields and productivities host cells that do not contain the heterologous nucleic acids.

Any suitable host cell may be used in practice of the methods of the present disclosure, and examples of host cells useful in the compositions and methods provided herein include archaeal, prokaryotic, or eukaryotic cells. In some embodiments of the present disclosure, the recombinant host cell is a prokaryotic cell. In some embodiments of the present disclosure, the recombinant host cell is a eukaryotic cell. In some embodiments of the present disclosure, the recombinant host cell is a Pichia kudriavzevii (P. kudriavzevii) strain. Methods of construction and genotypes of these recombinant host cells are described herein.

### Eukaryotic Cells

In some embodiments, eukaryotic cells are suitable for use in accordance with methods of the present disclosure, so long as the engineered host cell is capable of growth and/or product formation. Illustrative examples of eukaryotic host cells provided by the present disclosure include, but are not limited to, cells belonging to the genera Aspergillus, Crypthecodinium, Cunninghamella, Entomophthora, Mortierella, Mucor, Neurospora, Pythium, Schizochytrium, Thraustochytrium, Trichoderma, and Xanthophyllomyces. Examples of eukaryotic strains include, but are not limited to Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, Thraustochytrium aureum, Trichoderma reesei and Xanthophyllomyces dendrorhous. Illustrative examples of eukaryotic host cells provided by the present disclosure also include yeast cells, as detailed below.

### Yeast Cells

In some embodiments of the present disclosure, the recombinant host cell is a yeast cell. Yeast cells are excellent host cells for construction of recombinant metabolic pathways containing heterologous enzymes catalyzing production of small-molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Techniques for integration/insertion of nucleic acids into the yeast chromosome by homologous recombination are also well established. Yeast also offers a number of advantages as an industrial fermentation host. Yeast cells can generally tolerate high concentrations of organic acids and maintain cell viability at low pH and can grow under both aerobic and anaerobic culture conditions, and there are established fermentation broths and fermentation protocols. This characteristic results in efficient product biosynthesis when the host cell is supplied with a carbohydrate carbon source. Also, from a process standpoint, the ability to run fermentations under substantially anaerobic conditions can decrease production cost.

In various embodiments, yeast cells useful in the methods of the present disclosure include yeasts of the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In various embodiments, the yeast cell is of a species selected from the non-limiting group including Candida albicans, Candida ethanolica, Candida guilliermondii, Candida krusei, Candida lipolytica, Candida methanosorbosa, Candida sonorensis, Candida tropicalis, Candida utilis, Cryptococcus curvatus, Hansenula polymorpha, Issatchenkia orientalis, Kluyveromyces lactis, Kluyveromyces marxianus, Kluyveromyces thermotolerans, Komagataella pastoris, Lipomyces starkeyi, Pichia angusia, Pichia deserticola, Pichia galeiformis, Pichia kodamae, Pichia kudriavzevii (P. kudriavzevii), Pichia membranaefaciens, Pichia methanolica, Pichia pastoris, Pichia salicaria, Pichia stipins, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae (S. cerevisiae), Saccharomyces kluyveri, Schizosaccharomyces pombe (S. pombe) and Yarrowia lipolytica.

The Crabtree phenomenon refers to the capability of yeast cells to convert glucose to alcohol in the presence of high sugar concentrations and oxygen instead of producing biomass via the tricarboxylic acid (TCA) cycle. Yeast cells produce alcohol to prevent growth of competing microorganisms in high sugar environments, which yeast cells can utilize later on when the sugars are depleted. One skilled in the art will recognize that many yeast can typically use two pathways to produce ATP from sugars: the first involves the conversion of sugars (via pyruvate) to carbon dioxide via the TCA cycle, and the second involves the conversion of sugars (via pyruvate) to ethanol. Yeast cells that display a Crabtree effect (known as Crabtree-positive yeast cells) are able to simultaneously use both pathways. Yeast cells that do not display a Crabtree effect (known as Crabtree-negative yeast cells) will only convert pyruvate to ethanol at high yields when oxygen is absent.

In some embodiments of the present disclosure, the host cell is a Crabtree-positive yeast cell. In other embodiments, the host cell is a Crabtree-negative yeast cell. It may be advantageous to use a Crabtree-negative yeast to produce isobutyric acid because high glucose concentrations can be maintained during product biosynthesis without ethanol accumulation; ethanol is an undesired byproduct in isobutyric acid production. For example, P. kudriavzevii does not produce appreciable amounts of ethanol from pyruvate at high glucose concentrations in the presence of oxygen, and as such is a Crabtree-negative yeast. In some embodiments, the host cell is P. kudriavzevii.

In certain embodiments, the recombinant yeast cells provided herein are engineered by the introduction of one or more genetic modifications (including, for example, heterologous nucleic acids encoding enzymes and/or the disruption of native nucleic acids encoding enzymes) into a Crabtree-negative yeast cell. In certain of these embodiments, the host cell belongs to the Pichia/Issatchenkia/Saturnispora/Dekkera clade. In certain of these embodiments, the host cell belongs to the genus selected from the group including Pichia, Issatchenkia, or Candida. In certain embodiments, the host cell belongs to the genus Pichia, and in some of these embodiments the host cell is P. kudriavzevii. Members of the Pichia/Issatchenkia/Saturnispora/Dekkera or the Saccharomyces clade are identified by analysis of their 26S ribosomal DNA using the methods described by Kurtzman C. P., and Robnett C. J., (“Identification and Phylogeny of Ascomycetous Yeasts from Analysis of Nuclear Large Subunit (26S) Ribosomal DNA Partial Sequences”, Atonie van Leeuwenhoek 73(4):331-371; 1998). Kurtzman and Robnett report analysis of approximately 500 ascomycetous yeasts were analyzed for the extent of divergence in the variable D1/D2 domain of the large subunit (26S) ribosomal DNA. Host cells encompassed by a clade exhibit greater sequence identity in the D1/D2 domain of the 26S ribosomal subunit DNA to other host cells within the clade as compared to host cells outside the clade. Therefore, host cells that are members of a clade (e.g., the Pichia/Issatchenkia/Saturnispora/Dekkera or Saccharomyces clades) can be identified using the methods of Kurtzman and Robnett.

In certain embodiments of the present disclosure, the recombinant host cells are engineered by introduction of one or more genetic modifications into a Crabtree-positive yeast cell. In certain of these embodiments, the host cell belongs to the Saccharomyces clad. In certain of these embodiments, the host cell belongs to a genus selected from the group including Saccharomyces, Schizosaccharomyces, Brettanomyces, Torulopsis, Nematospora and Nadsonia. In certain embodiments, the host cell belongs to the genus Saccharomyces, and in one of these embodiments the host cell is S. cerevisiae.

### Prokaryotic Cells

In an embodiment of the present disclosure, the recombinant host cell is a prokaryotic cell. Prokaryotic cells are suitable host cells for construction of recombinant metabolic pathways including heterologous enzymes catalyzing production of small-molecule products. Prokaryotic cells may be archaeal cells or bacterial cells, as further detailed herein.

### Archaeal Cells

Archaeal cells are also suitable for use in accordance with methods of the present disclosure, and in some embodiments of the present disclosure, the recombinant host cell is an archaeal cell. Illustrative examples of recombinant archaea host cells provided by the present disclosure include, but are not limited to, cells belonging to the genera Aeropyrum, Archaeglobus, Halobacterium, Methanococcus, Methanobacterium, Pyrococcus, Sulfolobus, and Thermoplasma. Examples of archaea strains include, but are not limited to Archaeoglobus fulgidus, Halobacterium sp., Methanococcus jannaschii, Methanobacterium thermoautotrophicum, Thermoplasma acidophilum, Thermoplasma volcanium, Pyrococens horikoshii, Pyrococcus abyssi, and Aeropyrum pernix.

### Bacterial Cells

In an embodiment of the present disclosure, the recombinant host cell is a bacterial cell. Bacterial cells are suitable host cells for construction of recombinant metabolic pathways including heterologous enzymes catalyzing production of small-molecule products. Illustrative examples of recombinant bacterial host cells include, but are not limited to, cells belonging to the genera Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Pantoea, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas. Examples of bacterial strains include, but are not limited to, Bacillus subtilis (B. subtilis), Brevibacterium ammoniagenes, Bacillus amyloliquefacines, Brevibacterium ammoniagenes, Brevibacterium immariophilum, Clostridium acetobutylicum, Clostridium beigerinckii, Corynebacterium glutamicum (C. glutamicum), Enterobacter sakazakii, Escherichia coli (E. coli), Lactobacillus acidophilus, Lactococcus lactis, Mesorhizobium loti, Pantoea ananatis (P. ananatis), Pseudomonas aeruginosa, Pseudomonas mevalonii, Pseudomonas pudita, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus.

In some embodiments of the present disclosure, the recombinant host cell is selected from the group including Corynebacterium glutamicum, Escherichia coli, and Lactococcus lactis. In one embodiment, the recombinant host cell is Escherichia coli. In another embodiment, the recombinant host cell is Lactococcus lactis.

### Isobutyric Acid Pathway Enzymes

Provided herein in certain embodiments are recombinant host cells having at least one active isobutyric acid pathway from pyruvate to isobutyric acid. Recombinant host cells having an active isobutyric acid pathway as used herein produce active enzymes necessary to catalyze each metabolic reaction in an isobutyric acid pathway, and therefore are capable of producing isobutyric acid in measurable yields and/or titers when cultured under suitable conditions. Recombinant host cells having an active isobutyric acid pathway contain one or more heterologous nucleic acids encoding isobutyric acid pathway enzymes and are capable of producing isobutyric acid.

In certain embodiments, recombinant host cells of the present disclosure have an isobutyric acid pathway that proceeds via pyruvate (see, e.g. Table 1). This isobutyric pathway, as described herein, includes five enzymes: acetolactate synthase (EC 2.2.1.6), ketol-acid reductoisomerase (EC 1.1.1.86), dihydroxy-acid dehydratase (EC 4.2.1.9), branced-chain-2-oxoacid decarboxylase (EC 4.1.1.72) and isobutyraldehyde dehydrogenase (EC 1.2.1.5). In this pathway, all five isobutyric acid pathway enzymes are required to convert pyruvate to isobutyric acid.

TABLE 1
Enzymes Used To Convert Pyruvate to Isobutyric Acid

| EC # | Enzyme name | Reaction catalyzed |
|---|---|---|
| 2.2.1.6 | Acetolactate synthase | 2 Pyruvate → 2-Acetolactate + CO₂ |
| 1.1.1.86 | Ketol-acid | 2-Acetolactate + Reduced cofactor → 2,3- |
| | reductoisomerase | Dihydroxy-3-methylbutanoate + Oxidized cofactor |
| 4.2.1.9 | Dihydroxy-acid | 2,3-Dihydroxy-3-methylbutanoate → 3-Methyl-2- |
| | dehydratase | oxobutanoate + H₂O |
| 4.1.1.72 | Branched-chain-2-oxoacid | 3-Methyl-2-oxobutanoate → |
| | decarboxylase | Isobutyraldehyde + CO₂ |
| 1.2.1.5 | Isobutyraldehyde | Isobutyraldehyde + H₂O + Oxidized cofactor |
| | dehydrogenase | Isobutyric acid + Reduced cofactor |

One advantage of the described isobutyric acid pathway is that all five isobutyric acid pathway reactions are thermodynamically irreversible (i.e., have a negative Gibbs free energy such that greater than about 99% of reaction flux is calculated to be in the forward direction under physiological conditions—1 mM metabolite concentrations, 25° C., pH 7.0, and 0.1 M ionic strength—typically observed in a yeast) and three of the five isobutyric acid pathway reactions are catalyzed by unidirectional enzymes (i.e., where the enzyme mechanism does not permit the reverse reaction). Enzymes catalyzing either thermodynamically or mechanistically irreversible reactions are referred to herein as irreversible enzymes. As the pathway intermediates pass through these steps, they become locked into the portion of the isobutyric acid pathway downstream of each irreversible enzyme. Generally speaking, when multiple enzymes in a metabolic pathway are irreversible, this helps to increase product yields, titers, and productivities. A feature that is particularly advantageous in the described isobutyric acid pathway is that both the first and last two isobutyric acid pathway steps are thermodynamically and mechanistically irreversible; thus, there is a strong driving force both pushing carbon into the isobutyric acid pathway and out of the isobutyric acid pathway. In some embodiments of the present disclosure, the recombinant host cell contains an isobutyric acid pathway wherein some or all reaction steps are thermodynamically and/or mechanistically irreversible.

In addition to the specific isobutyric acid pathway steps being thermodynamically favored, the conversion of glucose to isobutyric acid is thermodynamically favored. The calculated cumulative change in Gibbs free energy at 1 mM metabolite concentrations, 25° C., pH 7.0, and 0.1 M ionic strength (i.e., ΔrG^{m}) for the conversion of glucose to isobutyric acid using the described isobutyric acid pathway is negative, and thus strongly favors product formation. The advantaged thermodynamics of the pathway will help to achieve high isobutyric acid yields, titers and productivities. The conversion of glucose to isobutyric acid using the isobutyric pathway described herein has a calculated ΔrG^{m} of −228.7+/−5.1 kJ/mol, indicative of a strong driving force that pushes the reaction to completion. Second, the pathway has net accumulation of 2 mol of ATP for every mol of isobutyric acid produced in the host cell cytosol from glucose, which can be used to help drive product export and support normal cell maintenance. Third, the balanced metabolic pathway requires a relatively low amount of oxygen (in fermentations where oxygen is used as the terminal electron acceptor) to sink excess NADH produced from pathway activity. In some embodiments of this disclosure, the recombinant host cell contains an isobutyric acid pathway providing a stoichiometric yield of 2 ATP per glucose converted to isobutyric acid in the host cell cytosol.

In certain embodiments, recombinant host cells may contain one or more heterologous nucleic acids encoding one, two, three, four, or all five, of the aforementioned isobutyric acid pathway enzymes or any combination thereof, wherein the heterologous nucleic acid is expressed in sufficient amounts to produce isobutyric acid.

In various embodiments, recombinant host cells may contain multiple copies of a single heterologous nucleic acid and/or multiple copies of two or more heterologous nucleic acids. Recombinant host cells including multiple heterologous nucleic acids may contain any number of heterologous nucleic acids.

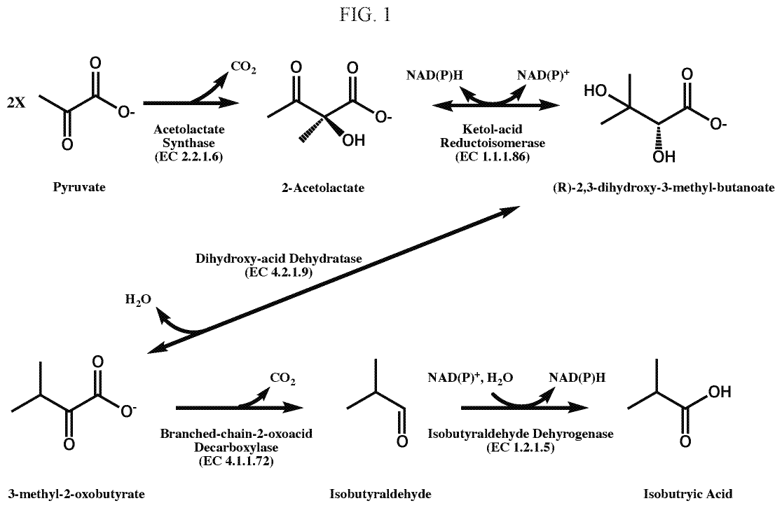

The recombinant host cells of the present disclosure may include recombinant host cells that employ combinations of metabolic reactions for biosynthetically producing the compounds of the present disclosure. The biosynthesized compounds can be produced intracellularly and/or secreted into the fermentation medium. The biosynthesized compounds produced by the recombinant host cells may include isobutyric acid, and the products and intermediates of the isobutyric acid pathway, namely acetolactate, 2,3-dihydroxy-3-methylbutanoate, 3-methyl-2-oxobutanoate, and isobutyraldehyde. The relationship of these compounds with respect to the metabolic reactions described herein is depicted in FIG. 1.

The present disclosure also provides consensus sequences useful in identifying and/or constructing isobutyric acid pathway enzymes suitable for use in accordance with the disclosed methods. An enzyme encompassed by a consensus sequence provided herein has an enzymatic activity that is identical, or essentially identical, or at least substantially similar with respect to ability to catalyze the reaction performed by one of the enzymes exemplified herein. Thus, for example, an acetolactate synthase encompassed by an acetolactate synthase consensus sequence provided herein has an enzymatic activity that is identical, or essentially identical, or at least substantially similar with respect to its ability to convert two molecules of pyruvate to one molecule acetolactate and one molecule CO₂.

Enzymes also useful in the compositions and methods provided herein include those that are homologous to consensus sequences provided by the disclosure. As noted above, any protein substantially homologous to an enzyme described herein can be used in a host cell of the disclosure. The percent sequence identity of an enzyme relative to a consensus sequence is determined by aligning the enzyme sequence against the consensus sequence. Those skilled in the art will recognize that various sequence alignment algorithms are suitable for aligning an enzyme with a consensus sequence. See, for example, Needleman, SB, et al, “A general method applicable to the search for similarities in the amino acid sequence of two proteins.” Journal of Molecular Biology 48 (3):443-53 (1970). Following alignment of the enzyme sequence relative to the consensus sequence, the percentage of positions where the enzyme possesses an amino acid described by the same position in the consensus sequence determines the percent sequence identity. In various embodiments, these consensus sequences may include active site amino acid residues believed to be necessary (although this disclosure is not to be limited by any theory of mechanism of action) for substrate recognition and reaction catalysis, as described below.

In addition to identification of useful enzymes by percent sequence identity with a given consensus sequence, enzymes useful in the compositions and methods provided herein can also be identified by the occurrence of highly conserved amino acid residues in the query protein sequence relative to a consensus sequence. For each consensus sequence provided herein, a number of highly conserved amino acid residues are described. Enzymes useful in the compositions and methods provided herein include those that may include a substantial number, and sometimes all, of the highly conserved amino acids at positions aligning with the indicated residues in the consensus sequence. Those skilled in the art will recognize that, as with percent identity, the presence or absence of these highly conserved amino acids in a query protein sequence can be determined following alignment of the query protein sequence relative to a given consensus sequence and comparing the amino acid found in the query protein sequence that aligns with each highly conserved amino acid specified in the consensus sequence.

### Acetolactate Synthase

Acetolactate synthase (ALS) (EC 2.2.1.6) as described herein catalyzes the conversion of two pyruvate molecules to 2-acetolacate and CO₂ (Table 1). Any enzyme is suitable for use in accordance with the disclosed methods so long as the enzyme is capable of catalyzing an ALS reaction. There are two types of acetolactate synthase (ALS) enzymes: anabolic ALS enzymes and catabolic ALS enzymes. Anabolic ALS enzymes are primarily found in plants, fungi, and bacteria, are involved in the biosynthesis of branched-chain amino acids, use a flavin adenine dinucleotide (FAD) cofactor, and are composed of multiple subunits, often including a regulatory subunit. By comparison, catabolic ALS enzymes are generally found only in some bacteria, have FAD-independent functionality, and lack a regulatory subunit. Either anabolic or catabolic ALSs are suitable for use in accordance with the methods of this disclosure. Generally speaking, catabolic ALSs are preferred since they do not require an FAD cofactor and do not possess a regulatory subunit.

### Catabolic Acetolactate Synthases

In many embodiments, the acetolactate synthase is derived from a bacterial source. In many of these embodiments, the acetolactate synthase is derived from a host cell belonging to a genus selected from the group including Bacillus, Enterobacter, and Klebsiella. In some embodiments, the acetolactate synthase is derived from Bacillus subtilis.

Non-limiting examples of catabolic ALS enzymes include those derived from Bacillus subtilis (UniProt ID: Q04789), Bacillus pumilus (UniProt ID: B4AJ14), Bacillus licheniformis (UniProt ID: T5HBH2), Bacillus amyloliquefaciens (UniProt ID: 12CAY3), Listeria innocua (UniProt ID: Q92A08), Bacillus pseudomycoides (UniProt ID: C3BGG6), Bacillus cereus (UniProt ID: C2Q7Y8), Bacillus cereus (UniProt ID: R8PL10), Bacillus thuringiensis (UniProt ID: C3DFV1), Enterobacter cloacae (UniProt ID: V3F760), and/or Klebsiella pneumoniae (UniProt ID: P27696).

In some embodiments, the ALS enzyme is the Bacillus subtilis AlsS protein (abbv. BsALSS; UniProt ID: Q04789; SEQ ID NO: 1).

In some embodiments, recombinant host cells may contain one or more heterologous nucleic acids encoding an ALS enzyme wherein the recombinant host cells are capable of producing isobutyric acid. In various embodiments, proteins suitable for use in accordance with methods of the present disclosure have ALS activity and may contain an amino acid sequence with at least 60%, at least 70%, at least 80%, at least 90%, or at least 95% sequence identity to SEQ ID NO: 1. In some embodiments, the recombinant host cell is a P. kudriavzevii strain.

Catabolic ALS Consensus Sequence #1; (SEQ ID NO: 19) provides the amino acid sequence of amino acids in which each position identifies the amino acid (if a specific amino acid is identified) or a subset of amino acids (if a position is identified as variable) most likely to be found at a specified position in a catabolic ALS enzyme. Those of skill in the art will recognize that fixed amino acids and conserved amino acids in these consensus sequences are identical to (in the case of fixed amino acids) or consistent with (in the case of conserved amino acids) with the wild-type sequence(s) on which the consensus sequence is based. Following alignment of a query protein with a consensus sequence provided herein, the occurrence of a dash (“−”) in the aligned query protein sequence indicates an amino acid deletion in the query protein sequence relative to the consensus sequence at the indicated position. Likewise, the occurrence of a plus (“+”) in the aligned consensus sequence indicates an amino acid addition in the query protein sequence relative to the consensus sequence at the indicated position. Amino acid additions and deletions are common to proteins encompassed by consensus sequences of this disclosure, and their occurrence is reflected as a lower percent sequence identity (i.e., amino acid addition or deletions are treated identically to amino acid mismatches when calculating percent sequence identity).

In various embodiments, ALS enzymes suitable for use in accordance with the methods of the present disclosure have ALS activity and may contain an amino acid sequence with at least 60%, at least 65%, or at least 70% sequence identity to SEQ ID NO: 19. For example, the BcALSS (SEQ ID NO: 1) sequence is 71% identical to consensus sequence SEQ ID NO: 19.

Many amino acids in SEQ ID NO: 19 are highly conserved and acetolactate synthases suitable for use in accordance with the methods of the present disclosure will include a substantial number, and sometimes all, of these highly conserved amino acids at positions aligning with the location of the indicated amino acid in SEQ ID NO: 19. The highly conserved amino acids in SEQ ID NO: 19 are G16, A17, L19, V21, L24, Q27, G28, V29, V32, F33, 135, P36, G37, A38, K39, 140, D41, V43, F44, D45, R58, H59, E60, N62, A63, A64, F65, M66, A67, A69, G71, R72, T74, G75, K76, G78, V79, L81, V82, T83, S84, G85, P86, G87, S89, N90, L91, T93, G94, T97, A98, E101, D103, P104, V105, V106, A107, G110, V112, R114, D116, K119, H122, Q123, S124, D126, A129, F131, P133, T135, K136, Y137, E140, V141, E149, N153, A154, F155, R156, A158, G164, S169, P171, Q172, D173, G193, A195, A210, P213, G218, R231, L233, P240, T244, Q246, A248, G249, G261, R262, G264, L265, F266, N268, Q269, G271, D272, L274, A278, D279, G285, P288, E290, Y291, P293, W296, N297, H306, D308, A312, Y318, P320, E323, L324, G326, I328, T331, H375, P376, L377, D396, G398, S399, I402, W403, R406, S419, N420, G421, Q423, T424, G426, V427, A428, L429, P430, W431, I433, A435, P440, K443, S446, S448, G449, D450, G451, G452, F453, L454, S456, M458, E459, L460, E461, T462, A463, V464, R465, H472, W475, D477, Y480, M482, V483, A484, Q486, K490, Y491, F499, G500, D503, A508, F511, G512, A513, G515, V518, L524, L528, G536, P537, P543, D545, Y546, D548, N549, and L552. In some embodiments, ALS enzymes suitable for use in accordance with the methods of this disclosure may include at least 60%, at least 70%, at least 80%, at least 85%, at least 90%, at least 95%, or sometimes all of these highly conserved amino acids at positions corresponding to (i.e., aligning with) the highly conserved amino acids identified in SEQ ID NO: 19. For example, SEQ ID NO: 1 includes 100% of these highly conserved amino acids at positions corresponding to their referenced positions in SEQ ID NO: 19. SEQ ID NO: 1 is a suitable ALS for use in accordance with the methods of the present disclosure. In some embodiments, each of these highly conserved amino acids are found in a desired ALS enzyme, as provided in SEQ ID NO: 19.

### Ketol-Acid Reductoisomerase

Ketol-acid reductoisomerases (KARI) (EC 1.1.1.86) are a family of oxidoreductases that produce intermediates in the biosynthesis of branched-chain amino acids. The KARI enzymes described herein catalyze the conversion of 2-acetolactate and NAD(P)H to 2,3-dihydroxy-3-methylbutanoate and NAD(P)⁺ (Table 1) and are used in the production of isobutyric acid in accordance with the methods of the present disclosure.

KARI enzymes typically utilize the cofactor NADP; NADPH is oxidized to NADP⁺ during catalysis. In most cell types, the pool of NAD (which consists of reduced and oxidized forms, i.e., NADH and NAD⁺, respectively) is larger than that of NADP. Under certain fermentation conditions, NADP may be even scarcer. Further, while interconversion of NADP with NAD can occur, the process is slow and inefficient. The limited availability and low regeneration rate of NADPH can hamper KARI enzyme turnover and isobutyric titers, yields or productivities during fermentation. Native KARI cofactor specificity can be altered, however, by standard microbial engineering techniques, and recombinant host cells can be designed to express modified KARI enzymes that utilize NADH, or NADH and NADPH non-selectively, instead of NADPH exclusively. The KARI enzymes of the present disclosure may include: 1) KARI enzymes that bind and oxidize NADH; 2) KARI enzymes that bind and oxidize NADPH; and/or 3) KARI enzymes that can indiscriminately bind and oxidize NADH and NADPH. In some embodiments of the present disclosure, the recombinant host cells may include a KARI enzyme that utilizes NADH as a cofactor and is capable of producing isobutyric acid. In some embodiments of the present disclosure, the recombinant host cell may include a KARI enzyme that utilizes NADPH as a cofactor and is capable of producing isobutyric acid. In some embodiments of the present disclosure, the recombinant host cell may include a KARI enzyme that utilizes NADH and/or NADPH as a cofactor and is capable of producing isobutyric acid.

Any enzyme is suitable for use in accordance with the methods of the present disclosure so long as the enzyme is capable of catalyzing the conversion of 2-acetolactate and NAD(P)H to 2,3-dihydroxy-3-methylbutanoate and NAD(P). In some embodiments, the KARI is derived from a bacterial source. In many of these embodiments, the KARI is derived from a host cell belonging to a genus selected from the group including Arcobacter, Bacillus, Bradyrhizobium, Campylobacter, Cellvibrio, Clostridium, Corynebacterium, Escherichia, Lactococcus, Leuconostoc, Mycobacterium, Propionibacterium, Pseudomonas, Ralstonia, Rhodococcus, Saccharopolyspora, Staphylococcus, Streptococcus, and Streptomyces. In one embodiment, the KARI is derived from Escherichia coli. In another embodiment, the KARI is derived from Lactococcus lactis.

Non-limiting examples of KARI enzymes include those derived from Arcobacter butzleri (UniProt ID: A8ERD8), Bacillus cereus (UniProt ID: Q81G13), Bacillus pumilus (UniProt ID: A8FFW6), Bacillus subtilis (UniProt ID: P37253), Bradyrhizobium diazoefficiens (UniProt ID: Q89G50), Campylobacter fetus (UniProt ID: A0RQ02), Cellvibrio japonicus (UniProt ID: B3PK17), Clostridium beijerinckii (UniProt ID: A6LPX8), Clostridium novyi (UniProt ID: A0Q0E9), Corynebacterium aurimucosum (UniProt ID: C3PFX1), Corynebacterium efficiens (UniProt ID: Q8FPX1), Corynebacterium glutamicum (UniProt ID: Q57179) Escherichia coli (UniProt ID: P05793), Lactococcus lactis (UniProt ID: Q02138), Leuconostoc mesenteroides (UniProt ID: Q03UU4), Mycobacterium smegmatis (UniProt ID: A0QUX8), Propionibacterium acnes (UniProt ID: Q6A7Z2), Pseudomonas fluorescens (UniProt ID: Q4K608), Pseudomonas putida (UniProt ID: Q88DZ0) Pseudomonas syringae (UniProt IDs: Q888N4 and Q4ZY66), Ralstonia solanacearum (UniProt ID: Q8XXN8), Rhodococcus jostii (UniProt ID: Q0S2H3), Saccharopolyspora erythraea (UniProt ID: A4FMQ5), Staphylococcus aureus (UniProt ID: A7X4M9), Staphylococcus brevis (UniProt ID: C2D2I9), Staphylococcus hominis (UniProt ID: A0A1L8Y8D1), Streptococcus gordonii (UniProt ID: A8AVN4), Streptococcus suis (UniProt ID: A4W3V8), and/or Streptomyces coelicolor (UniProt ID: Q9FBT8). In addition to the above listed bacterial KARI enzymes, suitable KARI enzymes can be derived from eukaryotic organisms. Most eukaryotic KARI enzymes are expressed in the mitochondria; however, expression of eukaryotic KARI comprising deletion of the N-terminal mitochondrial targeting sequences results in localization of the eukaryotic KARI to the host cell cytosol. A non-limiting example of a eukaryotic KARI suitable for use in producing isobutyric acid is S. cerevisiae mitochondrial Ilv5 (UniProt ID: P06168) comprising a deletion of the 47 N-terminal amino acids corresponding to the mitochondrial targeting sequence.

In a particular embodiment, the KARI enzyme is the E. coli IlvC protein (abbv. EcIL VC; UniProt ID: P05793; SEQ ID NO: 2). In other embodiments, the KARI is L. lactis IlvC (abbv. LlILVC; UniProt ID: Q02138; SEQ ID NO: 21).

In other embodiments, the KARI is selected from the group including Corynebacterium glutamicum IlvC (UniProt ID: Q57179), Lactococcus brevis IlvC (UniProt ID: C2D2I9), Lactococcus lactis IlvC (UniProt ID: Q02138), Pseudomonas fluorescens IlvC (UniProt ID: Q4K608), Pseudomonas putida IlvC (UniProt ID: Q88DZ0), Pseudomonas syringae IlvC (UniProt ID: Q4ZY66), Saccharomyces cerevisiae mitochondrial Ilv5 (UniProt ID: P06168 comprising a deletion of the 47 N-terminal amino acids), and Staphylococcus hominis IlvC (UniProt ID: A0A1L8Y8D1). As described in Example 9, expression of each of the KARI enzymes listed in this group in recombinant P. kudriavzevii expressing the other isobutyric acid pathway proteins resulted in detectable amounts of isobutyric acid.

In many embodiments, the recombinant host cell may contain one or more heterologous nucleic acids encoding a KARI enzyme wherein the recombinant host cells are capable of producing isobutyric acid. In other embodiments, recombinant host cells may contain one or more heterologous nucleic acids encoding a protein with KARI activity wherein the recombinant host cells are capable of producing isobutyric acid. In various embodiments, proteins suitable for use in accordance with methods of the present disclosure have KARI activity and may include an amino acid sequence with at least 60%, at least 70%, at least 80%, at least 90%, or at least 95% sequence identity to SEQ ID NO: 2. In many embodiments, the recombinant host cell is P. kudriavzevii strain.

SEQ ID NO: 20 represents a Class I KARI Consensus Sequence which was constructed based on the KARI enzyme sequences from Arcobacter butzleri, Bacillus cereus, Bacillus pumilus, Bacillus subtilis, Bradyrhizobium diazoefficiens, Campylobacter fetus, Cellvibrio japonicus, Clostridium beijerinckii, Clostridium novyi, Corynebacterium aurimucosum, Corynebacterium efficiens, Lactococcus lactis, Leuconostoc mesenteroides, Mycobacterium smegmatis, Propionibacterium acnes, Pseudomonas syringae, Ralstonia solanacearum, Rhodococcus jostii, Saccharopolyspora erythraea, Staphylococcus aureus, Streptococcus gordonii, Streptococcus suis, and Streptomyces coelicolor. The KARI enzymes from each of these organisms is a short-form (Class I) KARI generally found in fungi and most bacteria; the short-from KARI enzymes are distinguished from long-form (Class II) KARI enzymes by the absence of a central insert found in the Class II enzymes that is the structural equivalent of one C-terminal domain in a pair of a Class I KARI subunits.

In various embodiments, KARI enzymes suitable for use in accordance with the methods of the present disclosure have KARI activity and may include an amino acid sequence with at least 60%, at least 65%, or at least 70% sequence identity to SEQ ID NO: 20. For example, the LIILVC (SEQ ID NO: 21) sequence is 66% identical to consensus sequence SEQ ID NO: 20.

Many amino acids in SEQ ID NO: 20 are highly conserved and KARI enzymes suitable for use in accordance with the methods of the present disclosure may include many of these highly conserved amino acids at positions aligning with the location of the indicated amino acid in SEQ ID NO: 20. In certain cases, a substantial number of these highly conserved amino acids will align; in others, all of these highly conserved amino acids will align. The highly conserved amino acids in SEQ ID NO: 20 are Y7, D10, G25, G27, Q29, G30, H31, A32, L37, G41, V44, A58, G62, A75, M79, P83, D84, F107, H109, G110, A130, P131, K132, P134, G135, R139, G145, P149, L151, G160, G174, G179, T183, E188, D192, L193, F194, G195, E196, Q197, V199, L200, G202, G203, L207, G211, A218, G219, Y220, E223, A225, Y226, F227, E228, H231, E232, K234, I236, V237, D238, L239, G244, S253, G259, G264, M276, L280, Q284, G286, G319, and R323. In various embodiments, KARI enzymes homologous to SEQ ID NO: 20 include at least 60%, at least 70%, at least 80%, at least 85%, at least 90%, at least 95%, or sometimes all of these highly conserved amino acids at positions corresponding to the highly conserved amino acids identified in SEQ ID NO: 20. For example, SEQ ID NO: 21 (L1IL VC; UniProtID: Q02138) includes 100% of these highly conserved amino acids at positions corresponding to their referenced positions in SEQ ID NO: 20. SEQ ID NO: 21 is a suitable KARI for use in accordance with the methods of the present disclosure. In some embodiments, each of these highly conserved amino acids are found in a desired KARI enzyme, as provided in SEQ ID NO: 21.

### Dihydroxy-Acid Dehydratase

Dihydroxy-acid dehydratase (DHAD) (EC 4.2.1.9) described herein catalyzes the conversion of 2,3-dihydroxy-3-methylbutanoate to 3-methyl-2-oxobutanoate and water (Table 1). Any enzyme is suitable for use in accordance with the disclosed methods, so long as the enzyme is capable of catalyzing an DHAD reaction.

In some embodiments, the DHAD enzyme is derived from a bacterial source. In many of these embodiments, the DHAD is derived from a host cell belonging to a genus selected from the group including Arthrobacter, Acidovorax, Acinetobacter, Actinobacillus, Aeromonas, Agrobacterium, Bacillus, Bacteroides, Bifidobacterium, Burkholderia, Campylobacter, Chloroflexus, Clostridium, Corynebacterium, Lactococcus, Mycobacterium, Nocardioides, Polaromonas, Pseudoalteromonas, Rhodopseudomonas, Serratia, Staphylococcus, Stenotrophomonas, Streptococcus, Streptomyces, Vibrio, and Zymomonas. In one embodiment, the DHAD is derived from Lactococcus lactis.

Non-limiting examples of DHAD enzymes suitable for use in accordance with the methods of the present disclosure include those derived from Arthrobacter sp. (UniProt ID: A0JXZ9), Acidovorax citrulli (UniProt ID: A1TMA7), Acinetobacter baylyi (UniProt ID: Q6FCR9), Actinobacillus pleuropneumoniae (UniProt ID: A3MYG9), Aeromonas hydrophila (UniProt ID: A0KQS4), Agrobacterium radiobacter (UniProt ID: B9JDW2), Bacillus clausii (UniProt ID: Q5WEM9), Bacillus subtilis (UniProt ID: P51785), Bacteroides vulgatus (UniProt ID: A6L3E7), Bifidobacterium longum (UniProt ID: B7GUP9), Burkholderia lata (UniProt ID: Q39DS9), Campylobacter fetus (UniProt ID: A0RRN7), Chloroflexus aurantiacus (UniProt ID: A9WF68), Clostridium novyi (UniProt ID: A0Q0E8), Corynebacterium aurimucosum (UniProt ID: C3PFW7), Lactococcus lactis (UniProt ID: Q02139), Mycobacterium leprae (UniProt ID: O06069), Mycobacterium sp. (UniProt ID: A3PSS2), Nocardioides sp. (UniProt ID: A1SM84), Polaromonas sp. (UniProt ID: Q12BW0), Pseudoalteromonas haloplanktis (UniProt ID: Q3IJH1), Rhodopseudomonas palustris (UniProt ID: Q07IE7), Serratia proteamaculans (UniProt ID: A8GL60), Staphylococcus carnosus (UniProt ID: B9DMJ2), Staphylococcus saprophyticus (UniProt ID: Q49UX2), Stenotrophomonas maltophilia (UniProt ID: B4SMU1), Streptococcus suis (UniProt ID: A4W3W3), Streptomyces griseus (UniProt ID: B1VSL0), Vibrio cholerae (UniProt ID: A5F497), and/or Zymomonas mobilis (UniProt ID: Q5NLJ4).

In a particular embodiment, the DHAD enzyme is the Lactococcus lactis IlvD protein (abbv. L1ILVD; UniProt ID Q02139; SEQ ID NO: 3). In an embodiment of the present disclosure, recombinant host cells may contain one or more heterologous nucleic acids encoding a DHAD enzyme wherein the recombinant host cell is capable of producing isobutyric acid. In another embodiment, recombinant host cells may contain one or more heterologous nucleic acids encoding a protein with DHAD activity wherein the recombinant host cell is capable of producing isobutyric acid. In various embodiments, proteins suitable for use in accordance with methods of the present disclosure have DHAD activity and may include an amino acid sequence with at least 60%, at least 70%, at least 80%, at least 90%, or at least 95% sequence identity to SEQ ID NO: 3. In many embodiments, the recombinant host cell is a P. kudriavzevii strain.

SEQ ID NO: 22 represents a DHAD Consensus Sequence #1 which was constructed based on the DHAD enzyme sequences from Arthrobacter sp. (UniProt ID: A0JXZ9), Acidovorax citrulli (UniProt ID: A1TMA7), Acinetobacter baylyi (UniProt ID: Q6FCR9), Actinobacillus pleuropneumoniae (UniProt ID: A3MYG9), Aeromonas hydrophila (UniProt ID: A0KQS4), Agrobacterium radiobacter (UniProt ID: B9JDW2), Bacillus clausii (UniProt ID: Q5WEM9), Bacillus subtilis (UniProt ID: P51785), Bacteroides vulgatus (UniProt ID: A6L3E7), Bifidobacterium longum (UniProt ID: B7GUP9), Burkholderia lata (UniProt ID: Q39DS9), Campylobacter fetus (UniProt ID: A0RRN7), Chloroflexus aurantiacus (UniProt ID: A9WF68), Clostridium novyi (UniProt ID: A0Q0E8), Corynebacterium aurimucosum (UniProt ID: C3PFW7), Lactococcus lactis (UniProt ID: Q02139), Mycobacterium leprae (UniProt ID: O06069), Mycobacterium sp. (UniProt ID: A3PSS2), Nocardioides sp. (UniProt ID: A1SM84), Polaromonas sp. (UniProt ID: Q12BW0), Pseudoalteromonas haloplanktis (UniProt ID: Q3IJH1), Rhodopseudomonas palustris (UniProt ID: Q07IE7), Serratia proteamaculans (UniProt ID: A8GL60), Staphylococcus carnosus (UniProt ID: B9DMJ2), Staphylococcus saprophyticus (UniProt ID: Q49UX2), Stenotrophomonas maltophilia (UniProt ID: B4SMU1), Streptococcus suis (UniProt ID: A4W3W3), Streptomyces griseus (UniProt ID: B1VSL0), Vibrio cholerae (UniProt ID: A5F497), and Zymomonas mobilis (UniProt ID: Q5NLJ4).

In various embodiments, DHAD enzymes suitable for use in accordance with the methods of the present disclosure have DHAD activity and may include an amino acid sequence with at least 47%, at least 55%, at least 60%, at least 65%, or at least 70% sequence identity to SEQ ID NO: 22. For example, the LlILVD (SEQ ID NO: 3) sequence is 47% identical to consensus sequence SEQ ID NO: 22, and is therefore encompassed by consensus sequence SEQ ID NO: 22.

Many amino acids in SEQ ID NO: 22 are highly conserved and DHAD enzymes suitable for use in accordance with the methods of the present disclosure may include many of these highly conserved amino acids at positions aligning with the location of the indicated amino acid in SEQ ID NO: 22. In certain cases, a substantial number of these highly conserved amino acids will align; in others, all of these highly conserved amino acids will align. The highly conserved amino acids in SEQ ID NO: 22 are S6, G26, K34, P48, D81, G82, G89, M90, L94, S96, R97, I100, E105, D114, C122, D123, K124, P127, G128, G146, G151, G193, C195, G197, T200, A201, N202, E209, G212, A268, G276, G277, S278, N280, L283, H284, A287, P309, P316, G330, G331, G420, L425, G427, G433, K437, G450, A452, G482, P483, G485, P487, G488, M489, E491, M492, L493, T512, D513, G514, R515, S517, G518, G522, G526, H527, P530, E531, G536, D545, R568, A595, and G598. In various embodiments, DHAD enzymes homologous to SEQ ID NO: 22 contain at least 47%, at least 55%, at least 60%, at least 70%, at least 80%, at least 85%, at least 90%, at least 95%, or sometimes all of these highly conserved amino acids at positions corresponding to the highly conserved amino acids identified in SEQ ID NO: 22. For example, SEQ ID NO: 3 (LlILVD; UniProtID: Q02138) contains 100% of these highly conserved amino acids at positions corresponding to their referenced positions in SEQ ID NO: 22. SEQ ID NO: 3 is a suitable DHAD for use in accordance with the methods of the present disclosure. In some embodiments, each of these highly conserved amino acids are found in a desired DHAD enzyme, as provided in SEQ ID NO: 22.

### Branched-Chain-2-Oxoacid Decarboxylase

Branched-chain-2-oxoacid decarboxylase (OADC) (EC 4.1.1.72) described herein catalyzes the conversion of 3-methyl-2-oxobutanoate to isobutyraldehyde and CO2 (Table 1). Any enzyme is suitable for use in accordance with the disclosed methods so long as the enzyme is capable of catalyzing said conversion.

In some embodiments, the OADC enzyme is derived from a bacterial source. In many of these embodiments, the OADC is derived from a host cell belonging to a genus selected from the group including Carnobacterium, Enterococcus, and Lactococcus. In one embodiment, the OADC is derived from Lactococcus lactis.

Non-limiting examples of branched-chain-2-oxoacid decarboxylase include those derived from Carnobacterium maltaromaticum (UniProt ID: K8ENB2), Enterococcus caccae (UniProt ID: R3WVT4), Enterococcus haemoperoxidus (UniProt ID: R2SWI8), Enterococcus moraviensis (UniProt ID: R2QZ22), Influenza A virus (UniProt ID: A0A1X6), Lactococcus lactis KdcA (UniProt ID: Q6QBS4), Lactococcus lactis KivD (UniProt ID: Q684J7), Lactococcus lactis subsp. cremoris GE214 (UniProt ID: A0A084ABT7), Lactococcus lactis subsp. cremoris KW2 (UniProt ID: T2F5Q7), Lactococcus lactis subsp. lactis Dephy 1 (UniProt ID: U6ELQ5), Lactococcus lactis subsp. lactis IO-1 (UniProt ID: H5SZJ9), and Lactococcus lactis subsp. lactis KLDS 4.0325 (UniProt ID: U5PPW7).

In particular embodiments, the OADC enzyme is the Lactococcus lactis KivD protein (abbv. LlKIVD; UniProt ID Q684J7; SEQ ID NO: 4).

In some embodiments, recombinant host cells may include one or more heterologous nucleic acids encoding OADC wherein the recombinant host cells are capable of producing isobutyric acid. In other embodiments, recombinant host cells may include one or more heterologous nucleic acids encoding a protein with OADC activity wherein the recombinant host cells are capable of producing isobutyric acid. In various embodiments, enzymes suitable for use in accordance with methods of the present disclosure have OADC activity and contain an amino acid sequence with at least 60%, at least 70%, at least 80%, at least 90%, or at least 95% sequence identity to SEQ ID NO: 4. In many embodiments, the recombinant host cell is a P. kudriavzevii strain.

SEQ ID NO: 23 represents an OADC Consensus Sequence #1 which was constructed based on the OADC sequences from Carnobacterium maltaromaticum (UniProt ID: K8ENB2), Enterococcus caccae (UniProt ID: R3WVT4), Enterococcus haemoperoxidus (UniProt ID: R2SWI8), Enterococcus moraviensis (UniProt ID: R2QZ22), Influenza A virus (UniProt ID: A0A1X6), Lactococcus lactis KdcA (UniProt ID: Q6QBS4), Lactococcus lactis KivD (UniProt ID: Q684J7), Lactococcus lactis subsp. cremoris GE214 (UniProt ID: A0A084ABT7), Lactococcus lactis subsp. cremoris KW2 (UniProt ID: T2F5Q7), Lactococcus lactis subsp. lactis Dephy 1 (UniProt ID: U6ELQ5), Lactococcus lactis subsp. lactis IO-1 (UniProt ID: H5SZJ9), and Lactococcus lactis subsp. lactis KLDS 4.0325 (UniProt ID: U5PPW7).

In various embodiments, OADC enzymes suitable for use in accordance with the methods of the present disclosure have OADC activity and contain an amino acid sequence with at least 55%, at least 60%, at least 65%, at least 70%, at least 90%, or at least 95% sequence identity to SEQ ID NO: 23. For example, the LIKIVD (SEQ ID NO: 4) sequence is 95% identical to consensus sequence SEQ ID NO: 23.

Many amino acids in SEQ ID NO: 23 are highly conserved and OADC enzymes suitable for use in accordance with the methods of the present disclosure may contain many of these highly conserved amino acids at positions aligning with the location of the indicated amino acid in SEQ ID NO: 23. In certain cases, a substantial number of these highly conserved amino acids will align, in others, all of these highly conserved amino acids will align. The highly conserved amino acids in SEQ ID NO: 23 are Y2, T3, Y7, L8, L9, D10, R11, L12, E14, L15, G16, F21, G22, V23, P24, G25, D26, Y27, N28, L29, F31, L32, D33, W43, G45, N46, A47, N48, E49, L50, N51, A52, Y54, A56, D57, G58, Y59, A60, R61, T62, K63, A67, T70, T71, F72, G73, V74, G75, E76, L77, S78, A79, N81, G82, A84, G85, S86, A88, E89, P92, V93, 196, G98, P100, V104, Q105, K109, V111, H112, H113, T114, L115, D117, G118, F120, F123, A133, L137, N141, A142, E145, I146, D147, R148, V149, L150, P159, Y161, N163, L164, D167, A169, L180, L195, K197, L202, P208, G213, E215, S218, E222, P235, L239, G242, K243, E248, G255, Y257, G259, K267, V270, A273, D274, G280, L283, T284, D285, T288, F291, I301, L327, L354, Q356, W360, E364, T372, E376, Q377, G378, T379, S380, F381, F382, G383, K390, I396, G397, Q398, P399, L400, W401, G402, S403, I404, G405, T407, F408, P409, L412, G413, S414, Q415, A417, R422, H423, L424, L425, F426, I427, G428, D429, G430, S431, L432, Q433, L434, T435, Q437, E438, L439, G440, R444, K446, P449, F452, I454, N455, N456, G458, Y459, T460, V461, E462, R463, E464, I465, H466, G467, Y472, N473, D474, I475, P476, W478, Y480, L483, P484, F487, G488, V494, T501, E504, A512, D515, R518, W521, I522, E523, P532, L535, F542, A543, Q545, and N546. In various embodiments, OADC enzymes suitable for use in accordance with the methods of the present disclosure homologous to SEQ ID NO: 23 contain at least 60%, at least 70%, at least 80%, at least 85%, at least 90%, at least 95%, or sometimes all of these highly conserved amino acids at positions corresponding to the highly conserved amino acids identified in SEQ ID NO: 23. For example, SEQ ID NO: 4 (LlKIVD) contains 95% of these highly conserved amino acids at positions corresponding to their referenced positions in SEQ ID NO: 23. SEQ ID NO: 4 is a suitable OADC for use in accordance with the methods of the present disclosure. In some embodiments, each of these highly conserved amino acids are found in a desired OADC enzyme, as provided in SEQ ID NO: 23.

### Isobutyraldehyde Dehydrogenase

Isobutyraldehyde dehydrogenase (IBADH) (EC 1.2.1.5) described herein catalyzes the conversion of isobutyraldehyde, water and NAD(P)⁺ to isobutyric acid and NAD(P)H (Table 1).

Generally, IBADH enzymes are known to utilize the cofactor NADP; NADP⁺ is reduced to NADPH during catalysis. As with the above described KARI enzymes, in some organisms, dependence on NADP as a cofactor can hamper isobutyraldehyde dehydrogenase turnover and thus isobutyric acid titers, yields and/or productivities during fermentation. Using standard microbial engineering techniques, enzyme cofactor specificity can be modified and recombinant host cells can be designed to express isobutyraldehyde dehydrogenase with altered cofactor specificity. IBADH enzymes of the present disclosure may include: 1) IBADH enzymes that bind and reduce NAD⁺; 2) IBADH enzymes that bind and reduce NADP⁺; and/or 3) IBADH enzymes that can indiscriminately bind and reduce NAD⁺ and NADP⁺. In some embodiments of the present disclosure, recombinant host cells may contain an IBADH enzyme that utilizes NADH as a cofactor and is capable of producing isobutyric acid. In some embodiments, recombinant host cells may contain an IBADH enzyme that utilizes NADPH as a cofactor and is capable of producing isobutyric acid. In some embodiments, recombinant host cells may contain an IBADH enzyme that utilizes NADH and/or NADPH as a cofactor and is capable of producing isobutyric acid.

Any enzyme is suitable for use in accordance with the disclosed method so long as the enzyme is capable of catalyzing the conversion of isobutyraldehyde to isobutyric acid with concomitant reduction of NAD(P) to NAD(P)H. In some embodiments, the IBADH may be derived from a bacterial source. In many of these embodiments, the IBADH is derived from a host cell belonging to a genus selected from the group including Enterobacter, Escherichia, Gluconobacter, Klebsiella, Pseudomonas, Serratia, and Sphingobium. In one embodiment, the IBADH is derived from Escherichia coli.

Non-limiting examples of IBADH include those derived from Enterobacter asburide (UniProt ID: G2S1W8), Enterobacter lignolyticus (UniProt ID: E3G8V6), Enterobacteriaceae bacterium (UniProt ID: L0M324), Escherichia coli (UniProt ID: P80668), Gluconobacter thailandicus (UniProt ID: M9MLV8), Klebsiella pneumoniae (UniProt ID: A6T8G6), Pseudomonas sp. (UniProt ID: I4MZX3), Serratia marcescens (UniProt ID: L0MER4), and/or Sphingobium chlorophenolicum (UniProt ID: F6EWX4).

In a particular embodiment, the IBADH is the E. coli FeaB protein (abbv. EcFEAB; UniProt ID P80668; SEQ ID NO: 5).

In an embodiment of the present disclosure, recombinant host cells may include one or more heterologous nucleic acids encoding IBADH wherein the recombinant host cells are capable of producing isobutyric acid. In another embodiment, recombinant host cells may include one or more heterologous nucleic acids encoding a protein with IBADH activity wherein the recombinant host cells are capable of producing isobutyric acid. In various embodiments, proteins suitable for use in accordance with methods of the present disclosure have IBADH activity and may contain an amino acid sequence with at least 60%, at least 70%, at least 80%, at least 90%, or at least 95% sequence identity to SEQ ID NO: 5. In many embodiments, the recombinant host cell is a P. kudriavzevii strain.

SEQ ID NO: 24 represents an IBADH Consensus Sequence #1 which was constructed based on the isobutyraldehyde dehydrogenase sequences from Enterobacter asburide (UniProt ID: G2S1W8), Enterobacter lignolyticus (UniProt ID: E3G8V6), Enterobacteriaceae bacterium (UniProt ID: L0M324), Escherichia coli (UniProt ID: P80668), Gluconobacter thailandicus (UniProt ID: M9MLV8), Klebsiella pneumoniae (UniProt ID: A6T8G6), Pseudomonas sp. (UniProt ID: I4MZX3), Serratia marcescens (UniProt ID: L0MER4), and Sphingobium chlorophenolicum (UniProt ID: F6EWX4).

In various embodiments, IBADH enzymes suitable for use in accordance with the methods of the present disclosure may have IBADH activity and contain an amino acid sequence with at least 60%, at least 65%, or at least 70% sequence identity to SEQ ID NO: 24. For example, the EcFEAB (SEQ ID NO: 5) sequence is 92% identical to consensus sequence SEQ ID NO: 24.

Many amino acids in SEQ ID NO: 24 are highly conserved and IBADH enzymes suitable for use in accordance with the methods of the present disclosure may include many of these highly conserved amino acids at positions aligning with the location of the indicated amino acid in SEQ ID NO: 24. In certain cases, a substantial number of these highly conserved amino acids will align, in others, all of these highly conserved amino acids will align. The highly conserved amino acids in SEQ ID NO: 24 are F15, L16, R18, L22, G26, L37, P42, G45, I48, A52, D53, A54, D58, V59, A62, V63, S65, F70, W75, P80, A81, R83, E84, R85, L87, L88, R89, D92, L93, E95, E99, A102, Q103, L104, E105, L107, E108, Q109, G110, S112, I113, R117, E120, V121, W127, R129, Y130, A132, G133, L134, T136, K137, G140, T142, D144, S146, T159, E162, P163, G165, V166, V167, A168, P172, W173, N174, F175, P176, L177, I179, W182, K183, V184, P186, A187, L188, A189, G191, C192, S193, K197, P198, S199, E200, T202, P203, L204, T205, E211, A213, A216, G217, P219, G221, V222, F223, N224, T227, G228, G230, G234, L237, H240, P241, K245, S247, F248, T249, G250, S251, T252, G255, K256, I258, L271, E272, L273, G274, G275, K276, N277, P278, A279, L282, D284, A285, V290, G293, L294, F299, N301, G303, Q304, V305, C306, A307, A308, S310, R311, I312, Y313, E315, P317, D320, A329, G336, P337, G338, N346, P347, S350, H353, K356, L361, A364, A369, G374, P378, G382, Y384, P387, L389, N392, L399, R401, E403, V404, F405, G406, P407, V408, R413, V414, E418, L421, A424, N425, G430, L431, A433, S434, W436, T437, A443, A452, G453, T454, W456, V457, N458, H460, I463, D464, N466, P468, F469, G470, G471, K473, S475, G476, G478, R479, D480, F481, G482, W485, L486, D487, E491, K493, S494, and C496. In various embodiments, IBADH enzymes suitable for use in accordance with the methods of the present disclosure homologous to SEQ ID NO: 24 contain at least 60%, at least 70%, at least 80%, at least 85%, at least 90%, at least 95%, or sometimes all of these highly conserved amino acids at positions corresponding to the highly conserved amino acids identified in SEQ ID NO: 24. For example, SEQ ID NO: 5 (EcFEAB) includes 95% of these highly conserved amino acids at positions corresponding to their referenced positions in SEQ ID NO: 24. SEQ ID NO: 5 is a suitable IBADH for use in accordance with the methods of the present disclosure. In some embodiments, each of these highly conserved amino acids are found in a desired IBADH enzyme, as provided in SEQ ID NO: 24.

### Mitochondrial Isobutyic Acid Pathway Expression

The isobutyric acid pathway enzyme dihydroxyacid dehydratase (DHAD) uses a Fe—S cluster as a cofactor to catalyze a dehydration reaction. In eukaryotes, the Fe—S cluster biogenesis takes place in the mitochondria and most of the native Fe—S enzyme activity takes place in the mitochondria; while enzymes requiring the Fe—S cofactor are found in the yeast cytosol they are relatively rare. Yeast produces branched-chain amino acids (leucine, valine and isoleucine) using a mitochondrial pathway comprising the first three steps of the isobutyric acid pathway, namely acetolactate synthase (ILV2), ketol-acid reductoisomerase (ILV5), and the Fe—S utilizing enzyme dihydroxyacid dehydratase (ILV3). Thus, through the activity of these three enzymes yeast synthesize the isobutyric acid pathway intermediate 3-methyl-2-oxobutanoate. Expression of the isobutyric acid pathway enzymes in the yeast mitochondria can therefore take advantage of the higher occurrence of Fe—S cofactor in the mitochondria as compared to the cytosol, leading to higher DHAD activity and isobutyric acid production.

In some embodiments, the isobutyric acid pathway enzymes are expressed in the host cell cytosol resulting in isobutyric acid production. In other embodiments, the isobutyric acid pathway enzymes are expressed in the host cell mitochondria and result in isobutyric acid production. Those skilled in the art recognize that cytosolic proteins can be re-localized to the mitochondria when expressed in eukaryotic host cells (like yeast) by including an N-terminal mitochondrial targeting sequence. Various algorithms are readily available to identify suitable mitochondrial targeting sequences (see, for example, Fukasawa Y, et al., “MitoFates: improved prediction of mitochondrial targeting sequences and their cleavage sites.” Molecular and Cellular Proteomics 14(4):1113-1126 (2015)). One such mitochondrial targeting sequence in P. kudriavzevii is the 30 N-terminal amino acids of the mitochondrial pyruvate dehydrogenase complex protein PDA1 (UniProt ID: A0A099P5A5). As described in Example 10, expression of a mitochondrial isobutyric acid pathway comprising the same isobutyric acid pathway proteins expressed in the host cell cytosol, except with addition of a P. kudriavzevii PDA1 mitochondrial targeting sequence at each protein's N-terminus, resulted in isobutyric acid production.

### Ancillary Proteins

Ancillary proteins are other proteins that are expressed in recombinant host cells of the present disclosure whose expression results in an increase in isobutyric acid yields, productivities, and/or titers as compared to control, or host cells that do not express the proteins. Ancillary proteins function outside the isobutyric acid pathway, wherein each ancillary protein plays a role that increases the recombinant host cell's ability to produce isobutyric acid. Ancillary proteins may include any protein (excluding isobutyric acid pathway enzymes) of any structure or function that can increase isobutyric acid yields, titers, or productivities when expressed. Non-limiting examples of classes of ancillary proteins include transcription factors, transporters, scaffold proteins, proteins that decrease byproduct accumulation, and proteins that regenerate or synthesize redox cofactors. Ancillary proteins may be either native or non-native proteins. In cases where the protein is a native protein it is expressed in a non-native context, for example, it may be expressed from a heterologous nucleic acid.

Provided herein in certain embodiments are recombinant host cells containing one or more heterologous nucleic acids encoding one or more ancillary proteins wherein the recombinant host cell is capable of producing higher isobutyric acid yields, titers, or productivities as compared to control cells, or host cells that do not contain the heterologous nucleic acid(s).

In some embodiments, that host recombinant cell naturally produces isobutyric acid and in these cases, the isobutyric acid yields, titers, and/or productivities are increased following expression of one or more ancillary proteins. In other embodiments, the recombinant host cell does not natively produce isobutyric acid and has been engineered to produce isobutyric acid. In certain embodiments of the present disclosure, the recombinant host cells contain one or more heterologous nucleic acids encoding one or more isobutyric acid pathway enzymes and one or more heterologous nucleic acids encoding one or more ancillary proteins. In certain of these embodiments, the recombinant host cells may be engineered to express more of these ancillary proteins. In these particular embodiments, the ancillary proteins are expressed at a higher level (i.e., produced at a higher amount as compared to cells that do not express the ancillary proteins) and may be operatively linked to one or more exogenous promoters or other regulatory elements.

In certain embodiments, recombinant host cells contain both endogenous and heterologous nucleic acids encoding one or more isobutyric acid pathway enzymes and one or more ancillary proteins. In certain embodiments, the recombinant host cells contain one or more heterologous nucleic acids encoding one or more isobutyric acid pathway enzymes and/or one or more ancillary proteins, and one or more endogenous nucleic acids encoding one or more isobutyric acid pathway enzymes and/or one or more ancillary proteins.

In certain embodiments, endogenous nucleic acids of ancillary proteins may be modified in situ (i.e., on chromosome in the host cell genome) to alter levels of expression, activity, or specificity. In some embodiments, heterologous nucleic acids may be inserted into endogenous nucleic acids of ancillary proteins.

### Ancillary Proteins: Redox Cofactor Recycling

One category of ancillary proteins which may be used are proteins that recycle the redox cofactors produced during isobutyric acid pathway activity. Redox balance is fundamental to sustained metabolism and cellular growth in living organisms. Intracellular redox potential is determined by redox cofactors that facilitate the transfer of electrons from one molecule to another within a cell. Yeast-derived redox cofactors which may be used as ancillary proteins in the disclosed methods include the nicotinamide adenine dinucleotides, NAD and NADP, the flavin nucleotides, FAD and FMN, and iron sulfur clusters (Fe—S clusters). Redox cofactors may also be derived from other eukaryotic cells or derived from prokaryotic cells.

Redox constraints play an important role in end-product formation. Additional reducing power must be provided to produce compounds whose degree of reduction is higher than that of the substrate. Conversely, in the absence of oxygen as a terminal electron acceptor, producing compounds with a degree of reduction lower than that of the substrate will force the synthesis of byproducts with higher degrees of reduction to compensate for excess reducing power generated from substrate oxidation. Re-oxidation of NAD(P)H to NAD(P)⁺ is important for maintaining the thermodynamic driving force necessary for efficient and rapid isobutyric acid production and thus it is important to maintain redox neutrality to ensure efficient end-product formation. For example, the isobutyric acid pathway results in a net formation of 2 mol of NAD(P)H for each mol of isobutyric acid produced in the cytosol. In the absence of sufficient oxygen and/or electron transport chain flux, overflow metabolism (for example, in the yeast cytosol) can occur to reoxidize surplus cytosolic NAD(P)H, leading to byproduct accumulation and decreasing product titers, yields and/or productivities. Ancillary proteins can be expressed to reoxidize excess NAD(P)H without driving the formation of unwanted byproducts. Non-limiting examples of ancillary proteins that can be overexpressed to restore this redox balance include NADH dehydrogenase and water-forming NADH oxidase. In certain embodiments, the ancillary proteins may be expressed in the cytosol of recombinant host cells. In certain embodiments, the ancillary proteins may be associated with the mitochondrial or cell membrane of the recombinant host cells.

In many embodiments of the present disclosure, recombinant host cells capable of producing isobutyric acid may contain one or more nucleic acids encoding a NADH dehydrogenase ancillary protein. In some embodiments, the one or more nucleic acids are endogenous nucleic acids encoding a NADH dehydrogenase. In other embodiments, the one or more nucleic acids are heterologous nucleic acids encoding a NADH dehydrogenase. The yeast NADH dehydrogenase catalyzes the oxidation of NAD(P)H to NAD(P)⁺, thereby shuttling electrons from cytosolic NAD(P)H into the mitochondrial electron transport chain. Any NAD(P)H dehydrogenase can be used in accordance with the disclosed methods so long as it is capable of oxidizing NAD(P)H to NAD(P)⁺ in the cytosol. In many embodiments, the NADH dehydrogenase ancillary protein is expressed in the cytosol. In some embodiments, the NADH dehydrogenase is membrane associated or membrane bound. In some embodiments, the NADH dehydrogenase is a mitochondrial external NADH dehydrogenase. In yeast, the mitochondrial external NADH dehydrogenase is an inner-membrane mitochondrial protein with its catalytic site facing the intermembrane space. Because the mitochondrial outer membrane is permeable to most small molecules, the mitochondrial intermembrane space is considered to have the same environment as the cytosol. Therefore, the mitochondrial external NADH dehydrogenase contributes to the oxidation of cytosolic NAD(P)H and is a suitable ancillary protein for isobutyric acid production. In some embodiments, the mitochondrial external NADH dehydrogenase is the P. kudriavzevii Nde1 protein (abbv. PkNDE1; SEQ ID: NO 6). In some embodiments, recombinant host cells may contain one or more heterologous nucleic acids encoding one or more proteins suitable for use in accordance with methods of the present disclosure having NAD(P)H dehydrogenase activity and may further contain an amino acid sequence with at least 60%, at least 70%, at least 80%, at least 90%, or at least 95% sequence identity to SEQ ID NO: 6.

In some embodiments, recombinant host cells may contain one or more heterologous and/or endogenous nucleic acids encoding a water-forming NADH oxidase ancillary protein. The NADH oxidase converts NAD(P)H to NAD(P)⁺ and can restore redox balance in the host cell cytosol while reducing molecular oxygen, resulting in the formation of one mol water per mol NADH. Any NAD(P)H oxidase can be used in accordance with the disclosed methods so long as it is capable of oxidizing NAD(P)H to NAD(P)⁺ and reducing molecular oxygen to water in the cytosol.

In some embodiments, the water-forming NADH oxidase may be derived from a bacterial source. In many of these embodiments, the bacterial water-forming NADH oxidase is a Lactococcus lactis NoxE protein (abbv. L1NOXE; UniProt ID A2RIB7; SEQ ID: NO 7). In some embodiments, recombinant host cells may contain one or more heterologous nucleic acids encoding one or more proteins suitable for use in accordance with methods of the present disclosure have water-forming NAD(P)H oxidase activity and may contain an amino acid sequence with at least 60%, at least 70%, at least 80%, at least 90%, or at least 95% sequence identity to SEQ ID NO: 7.

In addition to being found in many bacteria, the water-forming NADH oxidases are also found in a number of archaeal microbes and in some embodiments, the NADH oxidase is derived from an archaeal source.

### Ancillary Proteins: Redox Cofactor Biogenesis

As explained in preceding paragraphs, redox balance is crucial for cell growth and sustained metabolism. Three out of the five isobutyric acid pathway enzymes detailed in Table 1 utilize redox cofactors that must be generated, in addition to being recycled, for robust metabolism and cell vitality. In some embodiments of the present disclosure, recombinant host cells may contain EcILVC that utilizes NAD(P)H. In some embodiments, recombinant host cells may contain LlILVD that utilizes Fe—S clusters. In some embodiments, recombinant host cells may contain EcFEAB that utilizes NAD(P). Thus, biogenesis and homeostasis of these cofactors are crucial for efficient catalysis of these enzymatic reactions.

Fe-S clusters facilitate various enzyme activities that require electron transfer. Because both iron and sulfur atoms are highly reactive and toxic to cells, Fe—S cluster assembly requires carefully coordinated synthetic pathways in living cells. The three known pathways are the Isc (iron sulfur cluster) system, the Suf (sulfur formation) system, and the Nif (nitrogen fixation) system. Each of these systems has a unique physiological role, yet several functional components are shared between them. First, a cysteine desulfurase enzyme liberates sulfur atoms from free cysteine. Then, a scaffold protein receives the liberated sulfur for Fe—S cluster assembly. Finally, the Fe—S cluster is transferred to a target apoprotein. In some embodiments of the present disclosure, recombinant host cells may contain heterologous and/or endogenous nucleic acids encoding one or more ancillary proteins that facilitate Fe—S cluster assembly. In some embodiments, the ancillary proteins may include one, more or all proteins of the Isc system, the Suf system, the Nif system, or any combination thereof. In some embodiments, recombinant host cells may contain one or more heterologous nucleic acids encoding one or more proteins suitable for use in accordance with methods of the present disclosure having cysteine desulfurase activity, Fe—S cluster assembly capability, Fe—S cluster transfer capability, iron chaperone capability, or any combination thereof.

Similar to Fe—S clusters, the NAD and NADP cofactors are involved in electron transfer and contribute to approximately 12% of all biochemical reactions in a cell (Osterman A., EcoSal Plus, 2009). NAD is assembled from L-aspartate, dihydroxyacetone phosphate (DHAP), phosphoribosyl pyrophosphate (PRPP) and ATP. The NADP is assembled in the same manner and further phosphorylated. In some embodiments, recombinant host cells may contain heterologous and/or endogenous nucleic acids encoding one or more ancillary proteins that facilitate NAD and NADP cofactor assembly. In some embodiments, the ancillary proteins may contain one, more or all proteins suitable for use in accordance with methods of the present disclosure having NAD and/or NADP assembly capability, NAD and/or NADP transfer capability, NAD and/or NADP chaperone capability, or any combination thereof.

### Ancillary Proteins: Isobutyric Acid Transport

Another class of ancillary proteins useful for increasing isobutyric acid yields, titers, and/or productivities are isobutyric acid transporter proteins. In some embodiments, recombinant host cells may contain one or more heterologous and/or endogenous nucleic acids encoding one or more isobutyric acid transporter proteins. In many embodiments, the isobutyric acid transporter is derived from a fungal source. In some embodiments, the isobutyric acid transporter may include those derived from S. cerevisiae PDR12 (abbv. ScPDR12; UniProt ID: Q02785), S. cerevisiae WAR1 (abbv. ScWAR1; UniProt ID: Q03631), and/or Kluyveromyces marxianus PDC12 (abbv. KmPDC12; UniProt ID: W0T9C6). In some embodiments, recombinant host cells may contain one or more heterologous nucleic acids encoding one or more proteins suitable for use in accordance with methods of the present disclosure have isobutyric acid transporter activity and may contain an amino acid sequence with at least 60%, at least 70%, at least 80%, at least 90%, or at least 95% sequence identity to ScPDR12, ScWAR1, and/or KmPDC12.

### Decreasing or Eliminating Expression of Byproduct Pathway Enzymes

In an additional aspect of this disclosure, nucleic acids encoding byproduct pathway enzymes may be disrupted in the recombinant host cells of the present disclosure to increase isobutyric acid yields, productivities, and/or titers; and/or to decrease byproduct titers and/or yields as compared to control cells, or host cells that express native/undisrupted levels of the byproduct pathway enzymes. Byproduct pathway enzymes may include any protein (excluding isobutyric acid pathway enzymes) of any structure or function that can decrease isobutyric acid yields, titers, or productivities when undisrupted because they utilize intermediates or products of the isobutyric acid pathway.

Byproducts that accumulate during isobutyric acid production and can lead to: 1) lower isobutyric acid titers, productivities and/or yields; and/or 2) accumulation of byproducts in the fermentation broth that increase the difficulty of downstream purification processes. In some embodiments, recombinant host cells may contain genetic disruptions that encompass alterations, deletions, substitutions, promoter modifications, premature stop codons and knock-outs, or knock-downs that decrease byproduct accumulation. In some embodiments, recombinant host cells containing a disruption of one or more genes encoding a byproduct pathway enzyme will have altered performance characteristics as compared to cells without the genetic disruption(s), such as decreased or eliminated byproduct pathway enzyme expression, decreased or eliminated byproduct accumulation, improved isobutyric acid pathway activity, altered metabolite flux through the isobutyric acid pathway, higher isobutyric acid titers, higher isobutyric acid productivities, higher isobutyric acid yields, and/or altered cellular fitness.

One important reason to decrease byproduct formation is that it allows an increase in isobutyric acid pathway activity, resulting in increased isobutyric acid production. In many embodiments, recombinant host cells of the present disclosure containing one or more genetic disruptions of one or more genes encoding a byproduct pathway enzyme may produce an increased isobutyric acid titer as compared to host cells that do not contain the genetic disruption(s). In some of these embodiments, the isobutyric acid titer in the fermentation broth is increased by 0.5 g/l, 1 g/1, 2.5 g/1, 5 g/1, 7.5 g/1, 10 g/l, or more than 10 g/l.

In addition to increasing isobutyric acid titers, decreasing byproduct formation can also help increase isobutyric acid yields. Because yield is independent of the volume of the fermentation broth, which can change during the course of a fermentation, it is often advantageous to measure isobutyric acid yields. In many embodiments, recombinant host cells of the present disclosure containing one or more genetic disruptions of one or more genes encoding byproduct pathway enzymes may produce an increased isobutyric acid yield as compared to host cells that do not contain the genetic disruption. In some of these embodiments, the isobutyric acid yield is increased by 0.5%, 1%, 2.5%, 5%, 7.5%, 10%, or more than 10% (g-isobutyric acid/g-substrate). The substrate in this yield calculation is the fermentation substrate, which is typically glucose, but may also be other, non-glucose substrates (e.g., sucrose, glycerol, or pyruvate).

Increasing isobutyric acid production is important for decreasing manufacturing costs, but it is also useful to disrupt genes encoding byproduct pathway enzymes in order to decrease byproduct formation. Byproducts are typically unwanted chemicals, are disposed of as waste, and their disposal can involve elaborate processing steps and containment requirements. Therefore, decreasing byproduct formation is generally also important for lowering production costs. In many embodiments, recombinant host cells of the present disclosure contain one or more genetic disruptions of one or more genes encoding a byproduct pathway enzyme may produce a lower byproduct titer as compared to host cells that do not contain the genetic disruption. In some of these embodiments, a recombinant host cell of the disclosure that contains genetic disruption of one or more byproduct pathway enzymes produces a byproduct titer that is 0.5 g/1, 1 g/l, 2.5 g/1, 5 g/1, 7.5 g/1, 10 g/l, or more than 10 g/l less than host cells that do not contain the genetic disruption.

In many embodiments, recombinant host cells of the present disclosure containing one or more genetic disruptions of one or more genes encoding a byproduct pathway enzyme produce a lower byproduct yield as compared to host cells that do not contain the genetic disruption(s). In some of these embodiments, recombinant host cells contain genetic disruption of one or more genes encoding byproduct pathway enzymes produce a byproduct yield that is 0.5%, 1%, 2.5%, 5%, 7.5%, 10%, or more than 10% (g-byproduct/g-substrate) less than host cells that do not contain the genetic disruption. As with the isobutyric acid yield calculation, the substrate used in the byproduct yield calculation is the carbon source provided to the fermentation; this is typically glucose, sucrose, or glycerol, but may be any carbon substrate.

Non-limiting examples of byproducts that arise due to consumption of an isobutyric acid pathway substrate, intermediate or product, include isobutanol, acetaldehyde, glycerone, carbon dioxide, acetaldehyde, acetoin, acetate, glycerol, and ethanol. In the event of a redox imbalance, an undesirable excess of reduced or oxidized cofactors may also accumulate; thus, the redox cofactors NADH, NAD⁺, NADPH and NADP⁺ can also be considered byproducts.

A non-limiting list of enzyme-catalyzed reactions that utilize the isobutyric acid pathway substrate (i.e., pyruvate), an isobutyric acid pathway intermediate, or isobutyric acid itself, are found in Table 2. Decreasing or eliminating expression of one, some or all of the genes encoding the enzymes in Table 2 can increase isobutyric acid production and/or decrease byproduct production. In many cases, the product of the enzyme-catalyzed reactions provided in Table 2 can accumulate in the fermentation broth; in such cases, this indicates that expression of the native gene encoding the listed enzyme should be reduced or eliminated. For example, the occurrence of dihydroxyacetone (also known as glycerone) in the fermentation broth indicates that expression of a native gene encoding glycerol dehydrogenase should be decreased or eliminated. In some cases, the product of the specific reaction listed in Table 2 is further converted, either spontaneously or through the action of other enzymes, into a byproduct that accumulates in the fermentation broth. For example, dihydroxyacetone is generally metabolized to glycerol, which is found to accumulate in the fermentation broth. In cases where byproduct accumulation is due to the activity of multiple enzymes, one or more of the genes encoding the one or more byproduct pathway enzymes can be deleted or disrupted to reduce byproduct formation.

In some embodiments of the present disclosure, recombinant host cells may be microbial strains with decreased or eliminated expression of one, some or all of the genes encoding enzymes listed in Table 2. In some embodiments, recombinant host cells may be microbial strains with decreased byproduct accumulation wherein the byproducts are formed through the activity of one, some or all of the enzymes listed in Table 2. In some embodiments, recombinant host cells may be microbial strains with decreased expression of pyruvate-utilizing enzymes. In some embodiments, recombinant host cells may be microbial strains with decreased expression of isobutyric acid-utilizing enzymes. In some embodiments, recombinant host cells may be microbial strains with inability to catabolize or breakdown isobutyric acid. In some embodiments, recombinant host cells may contain genetic modifications that reduce the ability of the host cells to catabolize the isobutyric acid pathway intermediates except via the isobutyric acid pathway. In some embodiments, recombinant host cells may contain genetic modifications that decrease the ability of the host cells to catabolize pyruvate except via the isobutyric acid pathway.

TABLE 2
Enzyme-catalyzed Reactions that Consume Isobutyric
Acid Pathway Substrate, Intermediate or Product

| Substrate | EC # | Enzyme name | Reaction formula |
|---|---|---|---|
| Isobutyrate | 6.2.1.1 | Isobutanoate/CoA-ligase | Isobutyrate + CoA + ATP → |
| | | | Isobutyryl-CoA + AMP + |
| | | | Diphosphate |
| Acetaldehyde | 1.2.1.3 | Acetaldehyde | Acetaldehyde + Oxidized |
| | | dehydrogenase | cofactor + H₂O → Acetate + |
| | | | Reduced cofactor + H⁺ |
| Aldehyde | 1.1.1.1 | Alcohol dehydrogenase | Aldehyde + Reduced cofactor → |
| | | | Alcohol + Oxidized cofactor |
| Isobutyraldehyde | 1.1.1.1 | Isobutyraldehyde | Isobutyraldehyde + Reduced |
| | | reductase | cofactor → Isobutanol + Oxidized |
| | | | cofactor |
| 2-Acetolactate | 4.1.1.5 | Acetolactate | 2-Acetolactate → Acetoin + CO₂ |
| | | decarboxylase | |
| Pyruvate | 1.1.1.28 | Lactate dehydrogenase | Pyruvate + Reduced cofactor + |
| | | | H⁺ [symbol] Lactate + Oxidized cofactor |
| Pyruvate | 4.1.1.1 | Pyruvate decarboxylase | Pyruvate + H⁺ → |
| | | | Acetaldehyde + CO₂ |
| Pyruvate | n/a | Pyruvate dehydrogenase | Pyruvate + CoA + Oxidized |
| | | complex | cofactor → Acetyl-CoA + CO₂ + |
| | | | Reduced cofactor |
| Pyruvate | 6.4.1.1 | Pyruvate carboxylase | Pyruvate + HCO₃⁻ + ATP → |
| | | | Oxaloacetate + ADP + |
| | | | Phosphate + H⁺ |

### Decreasing or Eliminating Expression of Pyruvate Decarboxylase

Pyruvate decarboxylase catalyzes the conversion of pyruvate to acetaldehyde and CO₂. This reaction is irreversible/unidirectional and, under most conditions, is thermodynamically favored over the acetolactate synthase catalyzed reaction (step 1 of the isobutyric acid pathway detailed in FIG. 1); the difference in equilibrium constant between acetolactate synthase and pyruvate decarboxylase, at 1 mM metabolite concentrations, i.e., ΔKeq′^{m}, is −8.97×10⁵ kJ/mol. Thus, under the calculated conditions of a recombinant host cell, pyruvate decarboxylase activity is favored (i.e., a negative ΔKeq′^{m}) over acetolactate synthase. Pyruvate decarboxylase activity can lead to the formation of at least three undesirable pyruvate decarboxylase-based byproducts: acetaldehyde, acetate, and ethanol. There are at least 3 pyruvate decarboxylase homologs in P. kudriavzevii: PkPDC1 (SEQ ID NO: 8), PkPDC5 (SEQ ID NO: 9) and PkPDC6 (SEQ ID NO: 10); decreasing or eliminating expression of one or more of these homologs is useful for increasing isobutyric acid production and/or decreasing accumulation of pyruvate decarboxylase-based byproducts. As described above, homologous proteins share substantial sequence identity with each other. Any protein that is homologous to any of the pyruvate decarboxylases of the present disclosure (SEQ ID NOs: 8, 9 and 10) will share substantial sequence identity one or more of these proteins.

In some embodiments, recombinant host cells may contain genetic disruptions in one or more pyruvate decarboxylase homologs. As defined above, genetic disruptions encompass nucleic acid deletions, nucleic acid insertions, nucleic acid substitutions, nucleic acid mutations, premature stop codons and promoter modifications. In some embodiments, recombinant host cells of the present disclosure may contain a genetic disruption of a homologous pyruvate decarboxylase gene with at least 60%, at least 70%, at least 80%, at least 90%, at least 95%, or more than 95% homology when compared to PkPDC1, PkPDC5 or PkPDC6. In some of these embodiments, the recombinant host cell is a P. kudriavzevii strain. In some embodiments, recombinant host cells may contain one or more gene disruptions that produce altered, decreased or eliminated activity in one, two or all three, pyruvate decarboxylase proteins. In some of these other embodiments, the recombinant host cell is a P. kudriavzevii strain.

In some embodiments, recombinant host cells may contain heterologous nucleic acids encoding isobutyric pathway enzymes, and may further contain one or more genetic disruptions of one, more, or all of the pyruvate decarboxylase homologs. In certain embodiments, acetaldehyde byproduct titer (i.e., g of byproduct/liter of fermentation volume) at the end of fermentation is 10 g/l or less, preferably 5 g/l or less, and most preferably 2.5 g/l or less. In certain embodiments, acetaldehyde byproduct yield (i.e., percentage of g of byproduct/g of substrate) at the end of fermentation is 10% or less, 5% or less, 2.5% or less, and preferably, 1% or less. In certain embodiments, acetate byproduct titer at the end of fermentation is 10 g/l or less, preferably 5 g/l or less, and most preferably 2.5 g/l or less. In certain embodiments, acetate byproduct yield at the end of fermentation is 10% or less, 5% or less, 2.5% or less, and preferably, 1% or less. In some embodiments, ethanol byproduct titer at the end of a fermentation is 10 g/l or less, preferably 5 g/l or less, and most preferably 2.5 g/l or less. In certain embodiments, ethanol byproduct yield at the end of fermentation is 10% or less, 5% or less, 2.5% or less, and preferably, 1% or less.

### Decreasing or Eliminating Expression of Pyruvate Dehydrogenase

The pyruvate dehydrogenase complex catalyzes the conversion of pyruvate, coenzyme A and NAD⁺ to acetyl-CoA, CO₂ and NADH; in wild type P. kudriavzevii, this enzyme is localized in the mitochondria. This reaction is irreversible/unidirectional and is thermodynamically favored over the acetolactate synthase (ALS) catalyzed reaction (step 1 of the isobutyric acid pathway); the ΔKeq′^{m} between ALS and pyruvate dehydrogenase complex is −8.48×10⁵ kJ/mol. Thus, pyruvate dehydrogenase complex activity is favored (i.e., a negative ΔKeq′^{m}) over ALS activity. In most native microbes, pyruvate dehydrogenase is used for aerobic metabolism of pyruvate to CO₂ through the activity of the tricarboxylic acid cycle enzymes. Genetic disruption of one or more genes encoding a protein of the pyruvate dehydrogenase complex can decrease pyruvate dehydrogenase complex protein activity or expression, consequently increasing isobutyric acid production and/or decreasing CO₂ byproduct formation. In some embodiments of the present disclosure, recombinant host cells may contain decreased or eliminated expression and/or activity of one or more pyruvate dehydrogenase complex proteins. In some of these embodiments, recombinant host cells may contain decreased or eliminated expression and/or activity of the E1 α-subunit of the pyruvate dehydrogenase complex (abbv. PkPDA1; SEQ ID NO: 11). In some embodiments, the recombinant host cell is a P. kudriavzevii strain.

### Decreasing or Eliminating Expression of Alcohol Dehydrogenase Enzymes

Alcohol dehydrogenase catalyzes the conversion of aldehyde and NAD(P)H to alcohol byproduct and NAD(P)⁺. Although many alcohol dehydrogenases are known to have high substrate specificity (e.g., are specific for the reduction of isobutyraldehyde to isobutanol), many others are substrate promiscuous and may reduce many different aldehyde substrates.

Multiple protein homologs with alcohol dehydrogenase activity may be present in a recombinant host cell. It may be necessary to genetically disrupt one or more alcohol dehydrogenase homologs to increase isobutyric acid pathway activity and/or decrease alcohol byproduct accumulation. In some embodiments of the present disclosure, recombinant host cells may contain heterologous nucleic acids encoding the isobutyric acid pathway enzymes and may further contain genetic disruptions of one or more alcohol dehydrogenase homologs. In some of these embodiments, the genetic disruptions give rise to decreased, altered or eliminated expression of one or more alcohol dehydrogenase homologs, and/or decreased, altered or eliminated activity of one or more alcohol dehydrogenase homologs.

In P. kudriavzevii, two alcohol dehydrogenase enzymes are the Adh1 protein (abbv. PkADH1; SEQ ID NO: 13) and the Adh6A protein (abbv. PkADH6A; SEQ ID NO: 14). In many embodiments, wherein the recombinant host cell is a P. kudriavzevii strain, recombinant host cells may contain genetic disruptions that give rise to decreased, altered or eliminated expression and/or activity of PkADH1 and/or PkADH6.

In some embodiments, recombinant host cells of the present disclosure may contain one or more genetic disruptions in one or more alcohol dehydrogenase homologs with at least 60%, at least 70%, at least 80%, at least 90%, at least 95%, or more than 95% homology when compared to PkADH1 and/or PkADH6A.

In some embodiments, recombinant host cells may contain heterologous nucleic acids encoding isobutyric pathway enzymes and may further contain one or more genetic disruptions of one or more alcohol dehydrogenase homologs. In certain embodiments, ethanol byproduct titer at the end of the fermentation is 10 g/l or less, preferably 5 g/l or less, and most preferably 2.5 g/l or less. In other embodiments, isobutanol byproduct yield at the end of fermentation is 10% or less, 5% or less, 2.5% or less, and preferably, 1% of less.

### Decreasing or Eliminating Expression of Acetaldehyde Dehydrogenase

Acetaldehyde dehydrogenase catalyzes the conversion of acetaldehyde, NAD(P)⁺ and water to acetate byproduct and NAD(P)H. Although many aldehyde dehydrogenases bind to a specific aldehyde substrate (such as acetaldehyde), many others are capable of binding indiscriminately to a variety of aldehyde substrates. In P. kudriavzevii, the acetaldehyde dehydrogenase proteins include the Ald2a protein (abbv. PkALD2A; SEQ ID NO: 15), the Ald2b protein (abbv. PkALD2B; SEQ ID NO: 16), the Ald3 protein (abbv. PkALD3; SEQ ID NO: 17), and the Ald6 protein (abbv. PkALD6; SEQ ID NO: 18). In some embodiments of the present disclosure, nucleic acids encoding PkALD2A, PkALD2B, PkALD3 or PkALD6, or any combination thereof, are genetically disrupted.

In embodiments of the present disclosure where in the recombinant host cell is a P. kudriavzevii strain, recombinant host cells may contain genetic disruptions that give rise to decreased, altered or eliminated expression or activity of PkALD2A, PkALD2B, PkALD3 or PkALD6, or any combination thereof. In some embodiments, recombinant host cells of the present disclosure may contain one or more genetic disruptions in one or more aldehyde dehydrogenase homologs with at least 60%, at least 70%, at least 80%, at least 90%, at least 95%, or more than 95% homology when compared PkALD2A, PkALD2B, PkALD3 or PkALD6.

In some embodiments, recombinant host cells may contain heterologous nucleic acids encoding isobutyric pathway enzymes, and may further contain one or more genetic disruptions of one, more, or all of the acetaldehyde dehydrogenase homologs. In certain embodiments, acetate byproduct titer at the end of fermentation 10 g/l or less, preferably 5 g/l or less, and most preferably 2.5 g/l or less. In certain embodiments, acetate byproduct yield at the end of fermentation is 10% or less, 5% or less, 2.5% or less, and preferably, 1% or less.

### Decreasing or Eliminating Expression of Glycerol-3-Phosphate Dehydrogenase

In addition to the possible byproducts derived from pyruvate and isobutyric acid pathway intermediates and product (as listed in Table 2), additional byproducts can arise from intermediates in glycolysis. Glycerol is a common byproduct that occurs under conditions of excess NADH. NAD-dependent glycerol-3-phosphate dehydrogenase catalyzes the conversion of dihydroxyacetone phosphate (glycerone phosphate) to glycerol-3-phosphate. NAD-dependent glycerol-3-phosphate dehydrogenase activity leads to the formation of the undesired byproduct glycerol. In P. kudriavzevii, NAD-dependent glycerol-3-phosphate dehydrogenase activity is encoded by the gene PkGPD1 (SEQ ID NO: 12). Decreasing or eliminating the expression of NAD-dependent glycerol-3-phosphate dehydrogenase is useful for decreasing glycerol byproduct accumulation. In some embodiments of the present disclosure, recombinant host cells may contain one or more genetic disruptions in one or more nucleic acids encoding a glycerol-3-phosphate dehydrogenase that gives rise to decreased, altered or eliminated expression and/or protein activity. In embodiments where the recombinant host cell is a P. kudriavzevii strain, the glycerol-3-phosphate dehydrogenase is PkGPD1. In some embodiments, recombinant host cells of the present disclosure may contain one or more genetic disruption(s) in one or more PkGPD1 homologs with at least 60%, at least 70%, at least 80%, at least 90%, at least 95%, or more than 95% homology when compared with PkGPD1.

In some embodiments, recombinant host cells may contain heterologous nucleic acids encoding isobutyric pathway enzymes and may further contain one or more genetic disruptions in one or more nucleic acids encoding glycerol-3-phosphate dehydrogenase. In certain embodiments, glycerol byproduct titer at the end of fermentation is 10 g/l or less, preferably at a titer of 5 g/l or less, and most preferably at a titer of 2.5 g/l or less. In certain embodiments, glycerol byproduct yield at the end of fermentation is 10% or less, 5% or less, 2.5% or less, and preferably, 1% or less.

### Genetic Engineering

Expression of isobutyric acid pathway enzymes in host cells can be achieved by transforming host cells with exogenous nucleic acids encoding isobutyric acid pathway enzymes, thus producing recombinant host cells of the present disclosure. The same is true for expression of ancillary proteins. Any method can be used to introduce exogenous nucleic acids into a host cell to produce a recombinant host cell of the present disclosure. Many such methods are known to practitioners in the art. Some examples include electroporation, transformation, conjugation and homologous recombination.

Recombinant host cells of the present disclosure may contain one or more exogenous nucleic acid molecules/elements, as well as single or multiple copies of a particular exogenous nucleic acid molecule/element as described herein. These molecules/elements may contain transcriptional promoters, transcriptional terminators, protein coding regions, open reading frames, regulatory sites, flanking sequences for homologous recombination, and intergenic sequences.

Exogenous nucleic acids can be maintained by recombinant host cells in various ways. In some embodiments, exogenous nucleic acids are integrated into the host cell genome. In other embodiments, exogenous nucleic acids are maintained in an episomal state that can be propagated, either stably or transiently, to daughter cells. Exogenous nucleic acids may include selectable markers to ensure propagation. In some embodiments, the exogenous nucleic acids are maintained in recombinant host cells with selectable markers. In some embodiments, the selectable markers are removed and exogenous nucleic acids are maintained in a recombinant host cell or strain without selection. In some embodiments, removal of selectable markers is advantageous for downstream processing and purification of the fermentation product.

In some embodiments, endogenous nucleic acids (i.e., genomic or chromosomal elements of a host cell), are genetically disrupted to alter, mutate, modify, modulate, disrupt, enhance, remove, or inactivate a gene product. In some embodiments, genetic disruptions alter expression or activity of proteins native to a host cell. In some embodiments, genetic disruptions circumvent unwanted byproduct formation or byproduct accumulation. Genetic disruptions occur according to the principle of homologous recombination via methods well known in the art. Disrupted endogenous nucleic acids can contain open reading frames as well as genetic material that is not translated into protein. In some embodiments, one or more marker genes replace deleted genes by homologous recombination. In some of these embodiments, the one or more marker genes are later removed from the chromosome using techniques known to practitioners in the art.

### Methods of Producing Isobutyric Acid

Methods are provided herein for producing isobutyric acid from recombinant host cells of the present disclosure. In certain embodiments, the methods include the steps of: 1) culturing a recombinant host cell as provided by the present disclosure in a fermentation broth containing at least one carbon source such that isobutyric acid is produced; and 2) recovering the isobutyric acid from the fermentation broth.

### Fermentative Production of Isobutyric Acid By Recombinant Host Cells

Any of the recombinant host cells of the present disclosure can be cultured to produce and/or secrete isobutyric acid. Materials and methods for the maintenance and growth of prokaryotic and eukaryotic cells, as well as fermentation conditions, are well known to practitioners of ordinary skill in the art. It is understood that consideration must be given to appropriate culture medium, pH, temperature, revival of frozen stocks, growth of seed cultures and seed trains, and requirements for aerobic, microaerobic, or anaerobic conditions, depending on the specific requirements of the host cells, the fermentation, and process flows.

The methods of producing isobutyric acid provided herein may be performed in a suitable fermentation broth in a suitable bioreactor such as a fermentation vessel, including but not limited to a culture plate, a flask, or a fermenter. Further, the methods can be performed at any scale of fermentation known in the art to support microbial production of small-molecules on an industrial scale. Any suitable fermenter may be used including a stirred tank fermenter, an airlift fermenter, a bubble column fermenter, a fixed bed bioreactor, or any combination thereof.

In some embodiments of the present disclosure, the fermentation broth is any fermentation broth in which a recombinant host cell capable of producing isobutyric acid according to the present disclosure, and can subsist (i.e., maintain growth, viability, and/or catabolize glucose or other carbon source). In some embodiments, the fermentation broth is an aqueous medium containing assimilable carbon, nitrogen, and phosphate sources. Such a medium can also include appropriate salts, minerals, metals, and other nutrients required for microbial growth and propagation. In some embodiments, the carbon source and each of the essential cell nutrients are provided to the fermentation broth incrementally or continuously, and each essential cell nutrient is maintained at essentially the minimum level required for efficient assimilation by growing cells. Examples of cell growth procedures include batch fermentation, fed-batch fermentation with batch separation, fed-batch fermentation with continuous separation, and continuous fermentation with continuous separation. These procedures are well known to practitioners of ordinary skill in the art.

In some embodiments of the present disclosure, the handling and culturing of recombinant host cells to produce isobutyric acid may be divided up into phases, such as growth phase, production phase, and/or recovery phase. The following paragraphs provide examples of features or purposes that may relate to these different phases. One skilled in the art will recognize that these features or purposes may vary based on the recombinant host cells used, the desired isobutyric acid yield, titer, and/or productivity, or other factors. While it may be beneficial in some embodiments for the isobutyric acid pathway enzymes, ancillary proteins and/or endogenous host cell proteins to be constitutively expressed, in other embodiments, it may be preferable to selectively express or repress any or all of the aforementioned proteins.

During growth phase, recombinant host cells may be cultured to focus on growing cell biomass by utilizing the carbon source provided. In some embodiments, the expression of isobutyric acid pathway enzymes and/or ancillary proteins is repressed or uninduced during growth phase. In some embodiments, no appreciable amount of isobutyric acid or any of its pathway intermediates is made. In some embodiments, proteins that contribute to cell growth and/or cellular processes may be selectively expressed.

During production phase, recombinant host cells may be cultured to stop producing cell biomass and to focus on isobutyric acid biosynthesis by utilizing the carbon source provided. In some embodiments, isobutyric acid pathway enzymes and/or ancillary proteins may be selectively expressed during the production phase to generate high product titers, yields and productivities. The production phase is synonymous with fermentation, fermentation run and/or fermentation phase.

In some embodiments, the growth and production phases take place at the same time. In other embodiments, the growth and production phases are separate. While in some embodiments, product is made exclusively during production phase, in other embodiments some product is also made during growth phase before production phase begins.

The recovery phase marks the end of the production phase, during which cellular biomass is separated from fermentation broth and isobutyric acid is purified from fermentation broth. Those skilled in the art will recognize that in some fermentation process, e.g., fill-draw and continuous fermentations, there may be multiple recovery phases where fermentation broth containing biomass and isobutyric acid are removed from the fermentation system. The draws of fermentation broth may be processed independently or may be stored, pooled, and processed together. In other fermentation processes, e.g., batch and fed-batch fermentations, there may only be a single recovery phase.

Fermentation procedures are particularly useful for the biosynthetic production of commercial isobutyric acid. It is understood by practitioners of ordinary skill in the art that fermentation procedures can be scaled up for manufacturing isobutyric acid and examples of fermentation procedures include, for example, fed-batch fermentation and batch product separation; fed-batch fermentation and continuous product separation; batch fermentation and batch product separation; and continuous fermentation and continuous product separation.

### Carbon Source for Fermentation

The carbon source provided to the fermentation can be any carbon source that can be fermented by the recombinant host cell. Suitable carbon sources include, but are not limited to, a variety of sugars including monosaccharides, disaccharides, polysaccharides, glycerol, acetate, ethanol, methanol, methane, or one or more combinations thereof. Examples of monosaccharides suitable for use in accordance to the methods of the present disclosure include, but are not limited to, dextrose (glucose), fructose, galactose, xylose, arabinose, and any combination thereof. Examples of disaccharides suitable for use in accordance to the methods of the present disclosure include, but are not limited to, sucrose, lactose, maltose, trehalose, cellobiose, and any combination thereof. Examples of polysaccharides suitable for use in accordance to the methods of the present disclosure include, but are not limited to, starch, glycogen, cellulose, and combinations thereof. In some embodiments, the carbon source is dextrose. In other embodiments, the carbon source is sucrose. In some embodiments, mixtures of some or all the aforementioned carbon sources can be used in fermentation.

### Fermentation pH

The pH of the fermentation broth can be controlled by the addition of acid or base to the culture medium. Preferably, fermentation pH is controlled at the beginning of fermentation and then allowed to drop as isobutyric acid accumulates in the broth, minimizing the amount of base added to the fermentation (thereby improving process economics) as well as minimizing the amount of salt formed. Specifically, the pH during fermentation is maintained in the range of 2-8, and more preferably, in the range of 4-8. At the end of fermentation, the final pH is in the range of 2-5. Non-limiting examples of suitable acids used to control fermentation pH include aspartic acid, acetate, hydrochloric acid, and/or sulfuric acid. Non-limiting examples of suitable bases used to control fermentation pH include sodium bicarbonate (NaHCO₃), sodium hydroxide (NaOH), potassium bicarbonate (KHCO₃), potassium hydroxide (KOH), calcium hydroxide (Ca(OH)₂), calcium carbonate (CaCO₃), ammonia, ammonium hydroxide, and/or diammonium phosphate. In some embodiments, a concentrated acid or concentrated base is used to limit dilution of the fermentation broth.

In some embodiments of the present disclosure, base is used for modulating pH and examples of base include NaHCO₃, NaOH, Ca(OH)₂, CaCO₃, NH₄, KHCO₃ and KOH. Base cations and isobutyrate anions react to form ionic compounds in fermentation broths. For example, base Na⁺ cations and isobutyrate anions react to form sodium butyrate. In some embodiments, the ionic compounds form by base cations and isobutyrate anions are soluble in fermentation broth. In other embodiments, the ionic compounds formed by base cations and isobutyrate anions are insoluble and may crystallize in the fermentation broth.

### Fermentation Temperature

The temperature of the fermentation broth can be any temperature suitable for growth of the recombinant host cells and/or production of isobutyric acid. Preferably, during isobutyric acid production, the fermentation broth is maintained within a temperature range of from about 20° C. to about 45° C., from about 25° C. to about 45° C., or from about 30° C. to about 42° C. In some embodiments, the fermentation broth is maintained at a temperature of about 20° C. In some embodiments, the fermentation broth is maintained at a temperature of about 25° C. In some embodiments, the fermentation broth is maintained at a temperature of about 30° C. In some embodiments, the fermentation broth is maintained at a temperature of about 35° C. In some embodiments, the fermentation broth is maintained at a temperature of about 40° C.

### Oxygen/Aeration

Generally speaking, microbial production of isobutyric acid from glucose results in the formation of NADH and/or NADPH, redox cofactors that must be converted back to NAD⁺ and NADP⁺ in order to maintain catabolismof glucose. Under aerobic conditions, microbes will commonly use molecular oxygen as an electron acceptor, enabling these cofactors to be reoxidized. If the fermentation is not appropriately oxygenated, isobutyric acid production can decrease. During cultivation, aeration and agitation conditions are selected to produce an oxygen transfer rate (OTR; amount of dissolved oxygen in a fermentation medium) that results in isobutyric acid formation. In various embodiments, fermentation conditions are selected to produce an OTR of greater than 10 mmol/l/hr. In some embodiment, fermentation conditions are selected to produce an OTR of greater than 20 mmol/l/hr, greater than 30 mmol/l/hr, greater than 40 mmol/l/hr, greater than 50 mmol/l/hr, greater than 75 mmol/l/hr, greater than 100 mmol/l/hr, greater than 125 mmol/l/hr, greater than 150 mmol/l/hr, greater than 175 mmol/l/hr, or greater than 200 mmol/l/hr. OTR as used herein refers to the volumetric rate at which oxygen is consumed during the fermentation. Inlet and outlet oxygen concentrations can be measured by exhaust gas analysis, for example by mass spectrometers. OTR can be calculated by one of ordinary skill in the art using the Direct Method described in Bioreaction Engineering Principles 3^{rd} Edition, 2011, Spring Science+Business Media, p. 449. The recombinant host cells of the present disclosure are able to produce isobutyric acid under a wide range of oxygen concentrations.

### Fermentation Yields and Titers

A high yield of isobutyric acid from the provided carbon source(s) is desirable to decrease the production cost. As used herein, yield is calculated as the percentage of the mass of carbon source catabolized by recombinant host cells of the present disclosure and used to produce isobutyric acid. In some cases, only a fraction of the carbon source provided to a fermentation is catabolized by the cells, and the remainder is found unconsumed in the fermentation broth or is consumed by contaminating microbes in the fermentation. Thus, it is important to ensure that fermentation is both substantially pure of contaminating microbes and that the concentration of unconsumed carbon source at the completion of the fermentation is measured. For example, if 100 grams of glucose is fed into the fermentation, and at the end of the fermentation 25 grams of isobutyric acid are produced and there remains 10 grams of glucose, the isobutyric acid yield is 27.7% (i.e., 25 grams isobutyric acid from 90 grams glucose). In certain embodiments of the methods provided herein, the final yield of isobutyric acid on the carbon source is at least 10%, at least 20%, at least 30%, at least 35%, at least 40%, or greater than 40%. In certain embodiments, the recombinant host cells provided herein are capable of producing at least 35%, at least 40%, or greater than 40% by weight of carbon source to isobutyric acid. Those skilled in the art will recognize that when an isobutyric acid salt (i.e., isobutyrate) is found in the fermentation broth the isobutyric acid yield can be determined by calculating the mols of isobutyric acid salt present and adjusting for the molecular weight difference between the isobutyric acid salt and isobutyric acid.

In addition to yield, the titer (or concentration), of isobutyric acid produced in the fermentation is another important metric for production. Assuming all other metrics are equal, a higher titer is preferred to a lower titer. Generally speaking, titer is provided as grams of product (e.g., isobutyric acid) per liter of fermentation broth (i.e., g/l). In some embodiments, the isobutyric acid titer is at least 1 g/l, at least 5 g/l, at least 10 g/l, at least 15 g/l, at least 20 g/l, at least 25 g/l, at least 30 g/l, at least 40 g/l, at least 50 g/l, at least 60 g/l, at least 70 g/l, at least 80 g/l, at least 90 g/l, at least 100 g/l, or greater than 100 g/l at some point during the fermentation, and preferably at the conclusion of the fermentation. As with yield calculations, those skilled in the art will recognize that an isobutyric acid titer can be calculated from the isobutyric acid salt titer by adjusting for molecular weight differences between the isobutyric acid salt and isobutyric acid.

Further, productivity, or the rate of product (i.e., isobutyric acid) formation, is important for decreasing production cost, and, assuming all other metrics are equal a higher productivity is preferred over a lower productivity. Generally speaking, productivity is provided as grams product produced per liter of fermentation broth per hour (i.e., g/l/hr). In some embodiments, isobutyric acid productivity is at least 0.1 g/l, at least 0.25 g/l, at least 0.5 g/l, at least 0.75 g/l, at least 1.0 g/l, at least 1.25 g/l, at least 1.25 g/l, at least 1.5 g/l, or greater than 1.5 g/l over some time period during the fermentation.

Practitioners of ordinary skill in the art understand that high-performance liquid chromatography (HPLC) is an appropriate method to determine the amount of isobutyric acid and/or isobutyric acid salts produced, the amount of any byproducts produced (e.g., organic acids and alcohols), the amount of any pathway metabolite or intermediate produced, and the amount of unconsumed glucose left in the fermentation broth. Aliquots of fermentation broth can be isolated for analysis at any time during fermentation, as well as at the end of fermentation. Briefly, molecules in the fermentation broth are first separated by liquid chromatography (LC); then, specific liquid fractions are selected for analysis using an appropriate method of detection (e.g., UV-VIS, refractive index, and/or photodiode array detectors). In some embodiments of the present disclosure, an organic acid salt (e.g., acetate and isobutyrate) is the fermentative product present in the fermentation broth. Practitioners in the art understand that the organic acid salt is acidified before or during HPLC analysis (producing acetic acid and isobutyric acid). Hence, the organic acid concentration calculated by HPLC analysis can be used to calculate the organic acid salt titer in the fermentation broth by adjusting for difference in molecular weight between the two compounds.

Gas chromatography is also an appropriate method to determine the amount of target product and byproducts, particularly if they are volatile. Samples of fermentation headspace can be isolated for analysis at any time during and after fermentation. Practitioners in the art understand that molecules are carried by an inert gas carries as they move through a column for separation and then arrive at a detector.

### EXAMPLES

### Methodology: Parent Strain

The parent strain in Example 1 was a P. kudriavzevii strain auxotrophic for histidine and uracil (i.e., the strain cannot grow in media without histidine and uracil supplementation). Histidine auxotrophy in the parent strain enables selection of new, engineered strains that carry a HIS3 marker, enabling histidine prototrophy and indicating desired strain construction. Likewise, uracil auxotrophy in the parent strain enables selection of new, engineered strains that carry a URA3 marker, enabling uracil prototrophy and indicating desired strain construction. Thus, cells that were successfully modified with exogenous nucleic acids to contain desired genetic modifications can grow in media without histidine and/or uracil supplementation, dependent on the selection marker included in the exogenous nucleic acid. Following confirmation of correct strain engineering, the selection marker(s) were removed by, for example, homologous recombination and marker loopout. Removing the marker enables subsequent rounds of strain engineering using the same selection markers.

### Methodology: Media

Complete supplement mixture (CSM) medium. CSM medium comprised Adenine 10 mg/L; L-Arginine HCl 50 mg/L; L-Aspartic Acid 80 mg/L; L-Histidine HCl 20 mg/L; L-Isoleucine 50 mg/L; L-Leucine 100 mg/L; L-Lysine HCl 50 mg/L; L-Methionine 20 mg/L; L-Phenylalanine 50 mg/L; L-Threonine 100 mg/L; L-Tryptophan 50 mg/L; L-Tyrosine 50 mg/L; Uracil 20 mg/L; L-Valine 140 mg/L. The YNB used in the CSM comprised Ammonium sulfate 5.0 g/L, Biotin 2.0 μg/L, Calcium pantothenate 400 μg/L, Folic acid 2.0 μg/L, Inositol 2.0 mg/L, Nicotinic acid 0-400 μg/L, p-Aminobenzoic acid 200 μg/L, Pyridoxine HCl 400 μg/L, Riboflavin 200 μg/L, Thiamine HCl 400 μg/L, Boric acid 500 μg/L, Copper sulfate 40 μg/L, Potassium iodide 100 μg/L, Ferric chloride 200 μg/L, Manganese sulfate 400 μg/L, Sodium molybdate 200 μg/L, Zinc sulfate 400 μg/L, Potassium phosphate monobasic 1.0 g/L, Magnesium sulfate 0.5 g/L, Sodium chloride 0.1 g/L, and Calcium chloride 0.1 g/L.

Complete supplement mixture minus histidine (CSM-His) medium. CSM-His medium is identical to CSM medium with the exception that histidine was not included in the medium. Engineered strains auxotrophic for histidine are unable to grow on CSM-His medium while engineered strains containing exogenous nucleic acids comprising a histidine selectable marker (e.g., HIS3) are capable of growth in CSM-His medium.

Complete supplement mixture minus uracil (CSM-Ura) medium. CSM-Ura medium is identical to CSM medium with the exception that uracil was not included in the medium. Engineered strains auxotrophic for uracil are unable to grow on CSM-Ura medium while engineered strains containing exogenous nucleic acids comprising a uracil selectable marker (e.g., URA3) are capable of growth in CSM-Ura medium.

BM02 medium. BM02 medium is Glucose 125 g/l, K₂SO₄ 0.816 g/l, Na₂SO₄ 0.1236, MgSO₄—7H₂O 0.304 g/l, Urea 4.3 g/l, Myo-inositol 2 mg/l, Thiamin HCl 0.4 mg/l, Pyridoxal HCl 0.4 mg/l, Niacin 0.4 mg/l, Ca-Pantothenate 0.4 mg/l, Biotin μg/l, Folic acid 2 μg/l, PABA 200 μg/l, Riboflavin 200 μg/l, Boric acid 0.25 mg/l, Copper sulfate pentahydrate 393 μg/l, Iron sulfate 11.0 mg/l, Manganese chloride 1.6 mg/l, Sodium molybdate 100 μg/l, Zinc sulfate 4 mg/l, and EDTA 11 mg/l.

BM02-P medium. BM02-P medium is BM02 medium with 1 g/l potassium phosphate.

YPE medium. YPE medium is Bacto peptone 20 g/l, Yeast extract 10g/l, and Ethanol 2%.

### Example 1: Construction of Recombinant P. kudriavzevii strain, LPK15779, with Eliminated Expression of Pyruvate Decarboxylase

This example describes the construction of a pyruvate decarboxylase (PDC) minus P. kudriavzevii, LPK15779, wherein all three PDC genes, i.e., Pdc1, Pdc5 and Pdc6, were genetically disrupted to eliminate expression of PkPDC1, PkPDC5, and PkPDC6.

The parent P. kudriavzevii strain used in this example was auxotrophic for uracil and histidine. To eliminate PDC expression, the Pdc1, Pdc5 and Pdc6 genes in the P. kudriavzevii genome were disrupted sequentially. The P. kudriavzevii strain was diploid and two copies of each pyruvate decarboxylase gene were present at the indicated locus; therefore, disruption of each gene was achieved by deleting both gene copies.

A URA3 selectable marker, amplified by PCR, was provided to the parent P. kudriavzevii strain to complement the uracil auxotrophic deficiency. The URA3 selectable marker contained unique upstream and downstream homologous regions for homologous recombination at the P. kudriavzevii Pdc1 locus, a transcriptional promoter, a URA3 coding region, and a transcriptional terminator. The transcriptional promoter 5′ of URA3 was the P. kudriavzevii TEF1 promoter (pPkTEF1) and the transcriptional terminator 3′ of URA3 was the S. cerevisiae TDH3 terminator (tScTDH3). The PCR product of the URA3 selectable marker was gel-purified and provided as exogenous nucleic acids to P. kudriavzevii. Transformation was carried out in a single step and gene deletion was achieved by homologous recombination. Transformants were selected on CSM-Ura medium and successful deletion of both copies of the gene encoding PkPDC1 was confirmed by genetic sequencing of this locus and the flanking regions. After successful construction of a recombinant P. kudriavzevii comprising a Pdc1 genetic disruption, the URA3 selectable marker was removed from the recombinant strain genome by recombination and marker loopout.

The URA3 selectable marker and genetic disruption strategy described above were reused to next disrupt the Pdc5 and Pdc6 genes in succession. Deletion of the native genes encoding PkPDC5 and PkPDC6 was confirmed by genetic sequencing of this locus and the flanking regions. The P. kudriavzevii strain that results from Example 1, LPK15779, was without any URA3 selectable marker. The URA3 selectable marker was absent in the following examples that describe further strain engineering or strain performance testing. Thus, Example 1 produces a PDC minus (i.e., contains deletion of native genes encoding PkPDC1, PkPDC5, and PkPDC6), uracil and histidine auxotrophic P. kudriavzevii, which was the background strain for Example 2 below.

### Example 2: Construction of Recombinant P. kudriavzevii Background Strain, LPK15942, with Eliminated Expression of Pyruvate Decarboxylase and Pyruvate Dehydrogenase Complex

This example describes the construction of a pyruvate dehydrogenase complex (PDH) minus P. kudriavzevii, LPK15942, wherein expression of PDH was eliminated via genetic disruption of the Pda1 gene. Pda1 encodes for the E1 α-subunit (PkPDA1) of the PDH. When PkPDA1 expression is eliminated, PDH cannot assemble into a functional complex. Thus, PDH expression is also eliminated and the recombinant host cell is unable to catalyze the conversion of pyruvate, coenzyme A and NAD⁺ to acetyl-CoA, CO₂ and NADH in the host cell mitochondria. This genetic disruption has the end result of decreasing respiration, thereby decreasing formation of byproduct CO₂ and increasing isobutyric acid production.

PkPDA1 was genetically disrupted using the same engineering strategy as described above in Example 1. LPK15779, a PDC minus, uracil and histidine auxotrophic P. kudriavzevii strain from Example 1 was the background strain used in Example 2.

A HIS3 selectable marker, amplified by PCR, was provided to the background strain (from Example 1) to complement the histidine auxotophic deficiency. The HIS3 selectable marker contained unique upstream and downstream homologous regions for homologous recombination at the Pda1 locus of the background strain genome, a transcriptional promoter, a HIS3 coding region, and a transcriptional terminator. The transcriptional promoter 5′ of HIS3 was the P. kudriavzevii TEF1 promoter (pPkTEF1) and the transcriptional terminator 3′ of HIS3 was the S. cerevisiae TDH3 terminator (tScTDH3). The PCR product of the HIS3 selectable marker was gel-purified and provided as exogenous nucleic acids to the background strain. Transformation was carried out in a single step and gene deletion was achieved by homologous recombination. Transformants were selected on CSM-His medium and successful deletion of both copies of the genes encoding PkPDA1 was confirmed by genetic sequencing of this locus and the flanking regions. After successful construction of a recombinant P. kudriavzevii comprising a Pda1 genetic disruption, the HIS3 selectable marker was removed from the recombinant strain genome by recombination and marker loopout.

The P. kudriavzevii strain that resulted from Example 2, LPK15942, was without a HIS3 selectable marker. The HIS3 selectable marker was absent in the following examples that describe further strain engineering or strain performance testing. Example 2 produced a PDC minus, PDH minus, uracil and histidine auxotrophic P. kudriavzevii (i.e., the strain contained deletion of native genes encoding PkPDC1, PkPDC5, PkPDC6, and PkPDA1), which was the background strain used in Example 3.

### Example 3: Recombinant P. kudriavzevii Background Strain, LPK15942, Does Not Naturally Produce Isobutyric Acid

This example describes the culturing and analysis of LPK15942 (from Example 2) for isobutyric acid production before LPK15942 was used as the background strain for genomic integration of the isobutyric acid pathway (Example 5, below). LPK15942 colonies were used to inoculate replicate tubes of 15 ml of YPE medium and incubated at 30° C. with 80% humidity and shaking at 250 rpm for 20 hours. These replicate tubes of pre-cultures were used to inoculate baffled flask replicates of 250 ml of BM02-P media with 10% glucose, 1% ethanol and 40 g/l CaCO₃. Pre-cultures were diluted 50×with 1 M HCl for OD₆₀₀ measurements to inform appropriate dilution of pre-cultures to produce a starting culture biomass of 1 g/l dry cell weight (DCW). Baffled flask cultures were then incubated at 30° C. with 80% humidity and shaking at 250 rpm. After 48 hours, the cultures were diluted 10×with 12 M HCl, spin-filtered and frozen for storage. Samples were analyzed by HPLC within 48 hours of harvest.

For HPLC analysis, frozen samples were thawed analyzed by HPLC using a Bio-Rad Aminex 87H column (300×7.8 mm) and a Bio-Rad Fermentation Monitoring column (#1250115; 150×7.8 mm) installed in series, with an isocratic elution rate of 0.8 ml/min with water at pH 1.95 (with sulfuric acid) at 30° C. Refractive index and UV 210 nm measurements were acquired for 35 minutes.

The LPK15942 background strain did not produce detectable amounts of isobutyric acid. Thus, all engineered P. kudriavzevii strains built from this background strain were incapable of producing isobutyric acid without the heterologous nucleic acids that encode the isobutyric acid pathway (Table 1 and Example 5).

### Example 4: Recombinant P. kudriavzevii Background Strain, LPK15942, Does Not Produce Significant Amounts of the Byproduct Ethanol

This example describes the culturing and analysis of LPK15942 (from Example 2) for basal level production of ethanol before LPK15942 was used as the background strain for genomic integration of the isobutyric acid pathway (Table 1 and Example 5). Native P. kudriavzevii cells are capable of producing the downstream metabolite ethanol via PDC-mediated decarboxylation of pyruvate to acetaldehyde. LPK15942 was a PDC minus strain incapable of decarboxylating pyruvate to acetaldehyde, and consequently incapable of producing ethanol. The PDC minus phenotype was constructed to provide the engineered isobutyric acid pathway access to the cellular pool of pyruvate without having to compete with PDC-mediated decarboxylation. LPK15942 should produce less ethanol than the PDC plus, wild type strain, designated LPK15, which was a P. kudriavzevii strain with all native PDC genes intact. LPK15942 and LPK15 were cultured and analyzed by HPLC according to methods described above, in Example 3.

The LPK15942 (PDC minus) background strain did not produce detectable amounts of ethanol while the LPK15 (PDC plus) strain produced greater than 50 mM of ethanol. This observation indicated that the PkPDC1, PkPDC5 and PkPDC6 genetic disruptions in LPK15942 produced a desired phenotype that did not produce excessive ethanol byproduct by PDC-mediated depletion of pyruvate. Thus, the cellular pool of pyruvate was available for the first reaction in the isobutyric acid pathway (Table 1).

### Example 5: Construction of Recombinant P. kudriavzevii Strains, LPK151338 and LPK151339, Expressing Isobutyric Acid Pathway Enzymes and Eliminating Expression of GPD1

This example describes the construction of recombinant host cells of the present disclosure that contain heterologous nucleic acids encoding isobutyric acid pathway enzymes (Table 1). The PDC minus, PDH minus, uracil and histidine auxotrophic P. kudriavzevii, LPK15942, from Example 2 was the background strain used in Example 5.

The heterologous nucleic acids used in this example encoded the following isobutyric acid pathway enzymes: B. subtilis AlsS (BsALSS; SEQ ID NO: 1), E. coli IlvC (EcILVC; SEQ ID NO: 2), L. lactis IlvD (LlILVD; SEQ ID NO: 3), L. lactis KivD (LlKIVD; SEQ ID NO: 4), and E. coli FeaB (EcFEAB; SEQ ID NO: 5). The genes encoding these proteins were codon-optimized for yeast and were synthesized and provided by Twist Bioscience; each gene was cloned into its own entry vector, pEV, along with an upstream transcriptional promoter and a downstream transcriptional terminator. The transcriptional promoters cloned in front (5′) of each gene were constitutive and derived from P. kudriavzevii. The promoters for BsALSS, EcILVC, LlILVD, LlKIVD, and EcFEAB were the PGK1 promoter (pPkPGK1), the ENO1 promoter (pPkENO1), the FBA1 promoter (pPkFBA1), the GPM1 promoter (pPkGPM1), and the TDH1 promoter (pPkTDH1), respectively. The transcriptional terminators cloned behind (3′) of each gene were derived from S. cerevisiae. The terminators for BsALSS, EcILVC, LlILVD, LlKIVD, and EcFEAB were the HXT1 terminator (tScHXT1), the TEF1 terminator (tScTEF1), the GRE3 terminator (tScGRE3), the PCY3 terminator (tScPYC2), and the TPI1 terminator (tScTPI1), respectively. Additionally, a HIS3 marker was included in the heterologous expression cassette to complement the histidine auxotrophic deficiency in the parent strain. This HIS3 marker contained a transcriptional promoter, a HIS3 coding region, and a transcriptional terminator. The transcriptional promoter 5′ of HIS3 was the P. kudriavzevii TEF1 promoter (pPkTEF1) and the transcriptional terminator 3′ of HIS3 was the S. cerevisiae TDH3 terminator (tScTDH3). Each gene was amplified from its respective pEV vector using primers with upstream and downstream homologous regions to neighboring genetic elements to drive correct assembly of the full-length pathway. The upstream and downstream homologous regions were 25 bp to 700 bp in length. The 5′ and 3′ ends of the expression cassette contained regions homologous to the genomic sequences upstream and downstream of the P. kudriavzevii GPD1 locus, thereby facilitating integration of the heterologous nucleic acids encoding the isobutyric acid pathway enzymes at the GPD locus in the P. kudriavzevii genome. Consequently, one or both copies of the PkGPD1 gene were deleted from the host genome; thus, genomic integration of the isobutyric acid pathway simultaneously decreased or eliminated expression of PkGPD1.

All PCR products were gel-purified and provided as exogenous nucleic acids to P. kudriavzevii. Transformation was carried out in a single step. Transformants were selected on CSM-His medium. Successful integration of all nucleic acids encoding isobutyric acid pathway enzymes as well as deletion of one or both copies of the genes encoding PkGPD1 were confirmed by genetic sequencing of this locus and the flanking regions

After successful construction of recombinant P. kudriavzevii host cells comprising isobutyric acid pathway genes, the HIS3 selectable markers were removed from recombinant host cell genomes by recombination and marker loopout. Example 3 produced recombinant host cells that contained heterologous nucleic acids encoding isobutyric acid pathway enzymes and additionally contained genetic disruption of PkPDC1, PkPDC5, PkPDC6, PkPDA1, and PkGPD1. The resulting strains were additionally auxotrophic for uracil and histidine. The recombinant host cell with one copy of PkGPD1 disrupted with a single genomic integration of the isobutyric acid pathway (i.e., a single GPD1 knockout) was designated LPK151338. The recombinant host cell with both copies of PkGPD1 disrupted with the isobutyric acid pathway genes (i.e., a double GPD1 knockout and a GPD minus phenotype) was designated LPK151339.

### Example 6: Construction of Recombinant P. kudriavzevii Strains, LPK151336 and LPK151337, with Single Or Double GPD1 Genetic Disruptions Via HIS3 Gene Integration at the GPD1 Locus

The same strain engineering strategy described in Example 5 was used to construct P. kudriavzevii strains with single or double GPD1 knockouts by genomic integration of the HIS3 selectable marker at the GPD1 locus. These strains were constructed to serve as GPD1 knockout control strains that lack the isobutyric acid pathway. In this example, the HIS3 selectable markers were not removed from recombinant host cell genomes. This example produced recombinant host cells that were auxotrophic for uracil and prototrophic for histidine, and further contained genetic disruption of PkPDC1, PkPDC5, PkPDC6, PkPDA1 and PkGPD1. The recombinant host cell with one copy of PkGPD1 disrupted with a single genomic integration of HIS3 (i.e., a single GPD1 knockout) was designated LPK151336. The recombinant host cell with both copies of PkGPD1 disrupted with HIS3 (i.e., a double GPD1 knockout and a GPD minus phenotype) was designated LPK151337.

### Example 7: Recombinant P. kudriavzevii Strains LPK151338 and LPK151339 Produce Isobutyric Acid Pathway Intermediate and Product

This example describes the culturing and analysis of recombinant host cells LPK151338 and LPK151339 from Example 5 (PDC minus, PDH minus, and single or double GPD1 knockout with single or double insertion of the isobutyric acid pathway), and LPK151336 and LPK151337 from Example 6 (PDC minus, PDH minus, and single or double GPD1 knock out with single or double insertion of the HIS3 gene) for in vivo production of the isobutyric acid pathway intermediate acetolactate, and product isobutyric acid (Table 1). All four recombinant strains were cultured and analyzed by HPLC according to methods described above in Example 3.

Both LPK151338 and LPG151339 produced about 6.5 mM to about 7.5 mM of isobutyric acid, and about 9.5 mM to about 10.5 mM of isobutyric acid, respectively. In contrast, the LPK15942 background strain did not produce detectable amounts of isobutyric acid (Example 3). Further, both LPK151338 and LKP151339 recombinant host cells produced about 10 g/l of acetolactate, which is the product of the first step in the isobutyric pathway (catalyzed by heterologous acetolactate synthase; Table 1). This example demonstrated, in accordance with the present disclosure, the expression of heterologous nucleic acids encoding isobutyric acid pathway enzymes (Table 1 and Example 5) in recombinant P. kudriavzevii for isobutyric acid production.

In addition, LPK151338 and LKP151339 produced about 165 mM to about 175 mM of acetoin, and about 170 mM to about 180 mM of acetoin, respectively. LPK151338 and LKP151339 contained a heterologous acetolactate synthase of the isobutyric acid pathway, enabling increased acetolactate production. Acetolactate can spontaneously convert to diacetyl, and acetolactate and/or diacetyl can migrate from the cytosol into the mitochondria, where the P. kudriavzevii native mitochondrial Bdh1 protein (PkBDH1) converts diacetyl to acetoin. Thus, increased amounts of acetolactate enabled increased acetoin production in the mitochondria. In contrast, the LPK151336 and LPK151337 strains, both PDC minus and PDH minus strains from Example 6 that lack the isobutyric acid pathway, produced about 15 mM to about 25 mM acetoin. Detection of increased acetoin levels in LPK151338 and LPK151339 in this example also demonstrated, in accordance with the present disclosure, the expression of heterologous nucleic acids encoding isobutyric acid pathway enzymes in recombinant P. kudriavzevii for isobutyric acid production.

### Example 8: Recombinant P. kudriavzevii Strains LPK151338 and LPK151339 Do Not Produce the Byproduct Glycerol

This example describes the culturing and analysis of LPK151338 (single GPD1 knockout) and LPK151339 (double GPD1 knockout; GPD minus) recombinant host cells (from Example 5) for in vivo production of glycerol. Native P. kudriavzevii cells are capable of producing the downstream metabolite glycerol via PkGPD1-mediated conversion of dihydroxyacetone phosphate (glycerone phosphate) to glycerol-3-phosphate. The presence of glycerol in fermentation media requires additional glycerol removal steps in isobutyric acid purification. The isobutyric acid pathway genes were inserted in the P. kudriavzevii GPD1 locus to disrupt expression of PkGPD1, thereby removing glycerol production in recombinant host cells. LPK151338 and LPK151339 were cultured and analyzed by HPLC according to methods described above in Example 3.

The single GPD1 knockout LPK151338 produced about 50 mM to about 110 mM of glycerol, while the double GPD1 knockout (hence, GPD1 minus) LPG151339 produced no detectable amounts of glycerol. This example demonstrates, in accordance with the present disclosure, the successful removal of glycerol from fermentation by genetic disruption of PkGPD1 in recombinant P. kudriavzevii for isobutyric acid production.

### Example 9: Recombinant P. kudriavzevii Strains Expressing KARI Enzymes

This example describes the construction, culturing, and analysis of recombinant host cells of the present disclosure that contain heterologous nucleic acids encoding KARI enzymes. The strains were constructed identically to LPK15338 from Example 5 with the exception that different KARI enzymes were used in place of the E. coli IlvC KARI protein. The KARI enzymes expressed in this example were Corynebacterium glutamicum IlvC (UniProt ID: Q57179), Lactococcus brevis IlvC (UniProt ID: C2D2I9), Lactococcus lactis IlvC (UniProt ID: Q02138), Pseudomonas fluorescens IlvC (UniProt ID: Q4K608), Pseudomonas putida IlvC (UniProt ID: Q88DZ0), Pseudomonas syringae IlvC (UniProt ID: Q4ZY66), Saccharomyces cerevisiae mitochondrial Ilv5 (UniProt ID: P06168 comprising a deletion of the 47 N-terminal amino acids corresponding to the mitochondrial targeting sequence), and Staphylococcus hominis IlvC (UniProt ID: A0A1L8Y8D1). The heterologous nucleic acids encoding the isobutyric acid pathway enzymes were integrated at the GPD1 locus, deleting one of the two native GPD1 alleles.

The recombinant P. kudriavzevii strains were cultured and analyzed by HPLC according to methods described above in Example 3. P. kudriavzevii strains LPK15942 (which does not produce isobutyric acid, see Example 3), and LPK15338 (which does produce isobutyric acid, see Example 7), were included as negative and positive controls, respectively. The negative control, LPK15942, produced an undetectable amount of isobutyric acid while the positive control, LPK15338, produced over 0.7 g/l isobutyric acid. The engineered strains comprising KARI enzymes derived from Corynebacterium glutamicum, Lactococcus brevis, Lactococcus lactis, Pseudomonas fluorescens, Pseudomonas putida, Pseudomonas syringae, Saccharomyces cerevisiae, and Staphylococcus hominis described here all produced isobutyric acid titers greater than 0.1 g/l. Thus, this Example demonstrates that, in accordance with the present disclosure, that any one of various KARI enzymes are suitable for use in engineered P. kudriavzevii for production of isobutyric acid.

### Example 10: Recombinant P. kudriavzevii Strains Producing Isobutyric Acid from a Mitochondrially Expressed Isobutyric Acid Pathway

This example describes the construction, culturing, and analysis of recombinant host cells of the present disclosure that express the isobutyric acid pathway proteins in the host cell mitochondria. The isobutyric acid pathway enzymes used in this example were Bacillus subtilis AlsS, Escherichia coli IlvC, Lactococcus lactis DHAD, Lactococcous lactis KivD, and Escherichia coli FeaB described in Example 5 with the exception that each protein included the mitochondrial targeting sequence from P. kudriavzevii PDA1 (i.e., the 30 N-terminal amino acids). The heterologous nucleic acids encoding this mitochondrial isobutyric acid pathway were inserted at the GPD1 locus in the P. kudriavzevii genome, deleting one of the two GPD1 alleles.

The recombinant P. kudriavzevii strains were cultured and analyzed by HPLC according to methods described above in Example 3. P. kudriavzevii strains LPK15942 (which does not produce isobutyric acid, see Example 3), and LPK15338 (which does produce isobutyric acid, see Example 7), were included as negative and positive controls, respectively. The negative control, LPK15942, produced an undetectable amount of isobutyric acid while the positive control, LPK15338, produced around 0.4 g/l isobutyric acid. The engineered strain expressing the mitochondrial isobutyric acid pathway produced around 1.2 g/l hour. Thus, this Example demonstrates that, in accordance with the present disclosure, that expression of a mitochondrial isobutyric acid pathway in P. kudriavzevii resulted in production of detectable amounts of isobutyric acid.

Various publications were referenced in this application. The disclosures of these publications in their entireties are hereby incorporated by reference in this application in order to more fully describe the state of the art to which this disclosure pertains.

It should be noted that there are alternative ways of implementing the embodiments disclosed herein. Accordingly, the present embodiments are to be considered as illustrative and not restrictive; various modifications can be made without departing from the spirit of the disclosure. Furthermore, the claims are not to be limited to the details given herein and are entitled their full scope and equivalents thereof.

What is claimed is:

1. A recombinant host cell, comprising:
an isobutyric acid biosynthetic pathway, comprising heterologous nucleic acids encoding an acetolactate synthase, a ketol-acid reductoisomerase, a dihydroxy-acid dehydratase, a branched-chain-2-oxoacid decarboxylase, and an isobutyraldehyde dehydrogenase;
wherein the heterologous nucleic acids are expressed in sufficient amounts to produce isobutyric acid.

2. The recombinant host cell of claim 1, which is selected from Pichia kudriazevii and Saccharomyces cerevisiae.

3. The recombinant host cell of claim 1, which is selected from Escherichia coli, Corynebacterium glutamicum, Bacillus subtilis, and Lactococcus lactis.

4. The recombinant host cell of any one of claims 1 to 3, wherein the acetolactate synthase has at least 60% amino acid identity to SEQ ID NO: 19 or SEQ ID NO: 1.

5. The recombinant host cell of claim 4, wherein the amino acid sequence of the acetolactate synthase is represented by SEQ ID NO: 1.

6. The recombinant host cell of any one of claims 1 to 5, wherein the ketol-acid reductoisomerase has at least 60% amino acid identity to SEQ ID NO: 20.

7. The recombinant host cell of any one of claims 1 to 5, wherein the ketol-acid reductoisomerase has at least 60% amino acid identity to SEQ ID NO: 2 or at least 60% amino acid identity to SEQ ID NO: 21.

8. The recombinant host cell of claim 7, wherein the amino acid sequence of the ketol-acid reductoisomerase is represented by SEQ ID NO: 2 or SEQ ID NO: 21.

9. The recombinant host cell of any one of claims 1 to 8, wherein the dihydroxy-acid dehydratase has at least 47% amino acid identity to SEQ ID NO: 22.

10. The recombinant host cell of claim 9, wherein the dihydroxy-acid dehydratase has at least 60% amino acid identity to SEQ ID NO: 3.

11. The recombinant host cell of claim 10, wherein the amino acid sequence of the dihydroxy-acid dehydratase is represented by SEQ ID NO: 3.

12. The recombinant host cell of any one of claims 1 to 11, wherein the branched-chain-2-oxoacid decarboxylase has at least 55% amino acid identity to SEQ ID NO: 23 or at least 60% amino acid identity to SEQ ID NO; 4.

13. The recombinant host cell of claim 12, wherein the amino acid sequence of the branched-chain-2-oxoacid decarboxylase is represented by SEQ ID NO: 4.

14. The recombinant host cell of any one of claims 1 to 13, wherein the isobutyraldehyde dehydrogenase has at least 55% amino acid identity to SEQ ID NO: 24 or at least 60% amino acid identity to SEQ ID NO: 5.

15. The recombinant host cell of claim 14, wherein the amino acid sequence of the isobutyraldehyde dehydrogenase is represented by SEQ ID NO: 5.

16. The recombinant host cell of any one of claims 1 to 15, further comprising:
one or more heterologous nucleic acids encoding one or more ancillary proteins; wherein the one or more ancillary proteins functions in redox cofactor recycling, redox cofactor biogenesis, or organic acid transport.

17. The recombinant host cell of claim 16, wherein the one or more ancillary proteins is mitochondrial external NADH dehydrogenase, water-forming NADH oxidase, isobutyric acid transporter, or combinations thereof.

18. The recombinant host cell of claim 17, wherein the mitochondrial external NADH dehydrogenase has at least 60% amino acid identity to SEQ ID NO: 6.

19. The recombinant host cell of claim 17 or claim 18, wherein the mitochondrial external NADH dehydrogenase is represented by SEQ ID NO: 6.

20. The recombinant host cell of any one of claims 17 to 19, wherein the water-forming NADH oxidase has at least 60% amino acid identity to SEQ ID NO: 7.

21. The recombinant host cell of claim 20, wherein the water-forming NADH oxidase is represented by SEQ ID NO: 7.

22. The recombinant host cell of any one of claims 17 to 21, wherein the isobutyric acid transporter has at least 60% amino acid identity to a protein selected from the group comprising Saccharomyces cerevisiae PDR12, Saccharomyces cerevisiae WAR1, and Kluyveromyces marxianus PDC12.

23. The recombinant host cell of any one of claims 1 to 22, further comprising a genetic disruption of one or more genes encoding pyruvate decarboxylase, pyruvate dehydrogenase, alcohol dehydrogenase, acetaldehyde dehydrogenase, glycerol-3-phosphate dehydrogenase, or combinations thereof.

24. The recombinant host cell of claim 23, wherein the one or more genes has at least 60% amino acid identity to SEQ ID NO: 12.

25. The recombinant host cell of claim 23, wherein the one or more genes encodes PkPDC1, represented by SEQ ID NO: 8, PkPDC5, represented by SEQ ID NO: 9, PkPDC6, represented by SEQ ID NO: 10, or combinations thereof.

26. The recombinant host cell of claim 23, wherein the one or more genes encodes PkPDA1, represented by SEQ ID NO: 11.

27. The recombinant host cell of claim 23, wherein the one or more genes encodes PkADH1, represented by SEQ ID NO: 13, PkADH6A, represented by SEQ ID NO: 14, or combinations thereof.

28. The recombinant host cell of claim 23, wherein the one or more genes encodes PkALD2A, represented by SEQ ID NO: 15, PkALD2B, represented by SEQ ID NO: 16, PkALD3, represented by SEQ ID NO: 17, PkALD6, represented by SEQ ID NO: 18, or combinations thereof.

29. A method for producing isobutyric acid, comprising:
culturing the recombinant host cell of any one of claims 1 to 28 to produce isobutyric acid.

30. The method of claim 29, wherein the culturing is performed at an oxygen transfer rate greater than 10 mmol/l/hr.

31. The method of claim 29 or claim 30, wherein the culturing is performed at a temperature of between about 25° C. and about 45° C.

32. The method of any one of claims 29 to 31, wherein the culturing is performed to produce a final fermentation broth pH of about pH 5.

33. An acetolactate synthase that has at least 60%, at least 70%, at least 80%, at least 90%, or at least 95% amino acid identity to SEQ ID NO: 19.

34. A ketol-acid reductoisomerase that has at least 60%, at least 70%, at least 80%, at least 90%, or at least 95% amino acid identity to SEQ ID NO: 21.

35. A dihydroxy-acid dehydatase that has at least 60%, at least 70%, at least 80%, at least 90%, or at least 95% amino acid identity to SEQ ID NO: 22.

36. A branched-chain-2-oxoacid decarboxylase that has at least 60%, at least 70%, at least 80%, at least 90%, or at least 95% amino acid identity to SEQ ID NO: 23.

37. An isobutyraldehyde dehydrogenase that has at least 60%, at least 70%, at least 80%, at least 90%, or at least 95% amino acid identity to SEQ ID NO: 24.


SEQUENCE LISTING (24 sequences)

SEQ ID NO: 1 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 2 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 3 - protein, 570 aa, Lactococcus lactis
MEFKYNGKVESVELNKYSKTLTQDPTQPATQAMYYGIGFKDEDFKKAQVGIVSMDWDGNPCNMHLGTLGSKIKSSVNQTDGLIGLQFHTIGVSDGIANGKLGMRYSLVSREVIADSIETNAGAEYYDAIVAIPGCDKNMPGSIIGMARLNRPSIMVYGGTIEHGEYKGEKLNIVSAFESLGQKITGNISDEDYHGVICNAIPGQGACGGMYTANTLAAAIETLGMSLPYSSSNPAVSQEKQEECDEIGLAIKNLLEKDIKPSDIMTKEAFENAITIVMVLGGSTNAVLHIIAMANAIGVEITQDDFQRISDITPVLGDFKPSGKYMMEDLHKIGGLPAVLKYLLKEGKLHGDCLTVTGKTLAENVETALDLDFDSQDIMRPLKNPIKATGHLQILYGNLAQGGSVAKISGKEGEFFKGTARVFDGEQHFIDGIESGRLHAGDVAVIRNIGPVGGPGMPEMLKPTSALIGAGLGKSCALITDGRFSGGTHGFVVGHIVPEAVEGGLIGLVEDDDIIEIDAVNNSISLKVSDEEIAKRRANYQKPTPKATRGVLAKFAKLTRPASEGCVTDL

SEQ ID NO: 4 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 5 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 6 - protein, 563 aa, Pichia kudriavzevii
MIPRLNPLLNISHLRGGPKFIGKAIKPSQFEFRKNNFRFNSTSTKTGSARTIKSGFLSWSFRAATFTGIAGWLYLTYLVYKETNPGSQSPQTEFSEIGNKKKNIVILGSGWGAVSVLKTLDTTKYNVTIVSPRNYFLFTPLLPSVPSGTIDIKSICDSIRTIARQTPGEVTYLEAAATDIDPVKKTIKLEHKSQRFLIGDAFTSEGDVIENELSYDYLVYAVGATVNTFGIPGIPEYASYLKEANDATAVRQKLFNQIEASRLLPKDSEDRKRLLSFVVCGGGPTGVELAAEIKDYIDQDLCKFIPGIEKEMQVTLIEAQHNVLSMFHPKLIEYTKEVFKQQNLHLQVDTMVKKVDDKNVYATYRHPDGKTEDMVIPYGTLVWAGGNAQRKLTRDLSSKIIEQKTARRGLLVDEYLKLDGDDSIYAIGDCTFTPNPPTAQVAHQQGEYLGEHFNKLAKIDELNYLITNSTDDSTKYSKRLERAEKAIKPFEYDHQGALAYVGSERAVADLHWGSWSTVALGGTMTFFFWRTAYVSMLLSIRNKILVVTDWVKVAIFGRDCSQE

SEQ ID NO: 7 - protein, 446 aa, Lactococcus lactis
MKIVVIGTNHAGIATANTLIDRYPGHEIVMIDRNSNMSYLGCGTAIWVGRQIEKPDELFYAKAEDFEKKGVKILTETEVSEIDFTNKMIYAKSKTGEKITESYDKLVLATGSRPIIPNLPGKDLKGIHFLKLFQEGQAIDEEFAKNDVKRIAVIGAGYIGTEIAEAAKRRGKEVLLFDAESTSLASYYDEEFAKGMDENLAQHGIELHFGELAQEFKANEKGHVSQIVTNKSTYDVDLVINCIGFTANSALAGEHLETFKNGAIKVDKHQQSSDPDVSAVGDVATIYSNALQDFTYIALASNAVRSGIVAGHNIGGKSIESVGVQGSNGISIFGYNMTSTGLSVKAAKKIGLEVSFSDFEDKQKAWFLHENNDSVKIRIVYETKNRRIIGAQLASKSEIIAGNINMFSLAIQEKKTIDELALLDLFFLPHFNSPYNYMTVAALNAK

SEQ ID NO: 8 - protein, 575 aa, Pichia kudriavzevii
MTDKISLGTYLFEKLKEAGSYSIFGVPGDFNLALLDHVKEVEGIRWVGNANELNAGYEADGYARINGFASLITTFGVGELSAVNAIAGSYAEHVPLIHIVGMPSLSAMKNNLLLHHTLGDTRFDNFTEMSKKISAKVEIVYDLESAPKLINNLIETAYHTKRPVYLGLPSNFADELVPAALVKENKLHLEEPLNNPVAEEEFIHNVVEMVKKAEKPIILVDACAARHNISKEVRELAKLTKFPVFTTPMGKSTVDEDDEEFFGLYLGSLSAPDVKDIVGPTDCILSLGGLPSDFNTGSFSYGYTTKNVVEFHSNYCKFKSATYENLMMKGAVQRLISELKNIKYSNVSTLSPPKSKFAYESAKVAPEGIITQDYLWKRLSYFLKPRDIIVTETGTSSFGVLATHLPRDSKSISQVLWGSIGFSLPAAVGAAFAAEDAHKQTGEQERRTVLFIGDGSLQLTVQSISDAARWNIKPYIFILNNRGYTIEKLIHGRHEDYNQIQPWDHQLLLKLFADKTQYENHVVKSAKDLDALMKDEAFNKEDKIRVIELFLDEFDAPEILVAQAKLSDEINSKAA

SEQ ID NO: 9 - protein, 608 aa, Pichia kudriavzevii
MLQTANSEVPNASQITIDAASGLPADRVLPNITNTEITISEYIFYRILQLGVRSVFGVPGDFNLRFLEHIYDVHGLNWIGCCNELNAAYAADAYAKASKKMGVLLTTYGVGELSALNGVAGAYTEFAPVLHLVGTSALKFKRNPRTLNLHHLAGDKKTFKKSDHYKYERIASEFSVDSASIEDDPIEACEMIDRVIYSTWRESRPGYIFLPCDLSEMKVDAQRLASPIELTYRFNSPVSRVEGVADQILQLIYQNKNVSIIVDGFIRKFRMESEFYDIMEKFGDKVNIFSTMYGKGLIGEEHPRFVGTYFGKYEKAVGNLLEASDLIIHFGNFDHELNMGGFTFNIPQEKYIDLSAQYVDITGNLDESITMMEVLPVLASKLDSSRVNVADKFEKFDKYYETPDYQREASLQETDIMQSLNENLTGDDILIVETCSFLFAVPDLKVKQHTNIILQAYWASIGYALPATLGASLAIRDFNLSGKVYTIEGDGSAQMSLQELSSMLRYNIDATMILLNNSGYTIERVIVGPHSSYNDINTNWQWTDLLRAFGDVANEKSVSYTIKEREQLLNILSDPSFKHNGKFRLLECVLPMFDVPKKLGQFTGKIPA

SEQ ID NO: 10 - protein, 584 aa, Pichia kudriavzevii
MAPVSLETCTLEFSCKLPLSEYIFRRIASLGIHNIFGVPGDYNLSFLEHLYSVPELSWVGCCNELNSAYATDGYSRTIGHDKFGVLLTTQGVGELSAANAIAGSFAEHVPILHIVGTTPYSLKHKGSHHHHLINGVSTREPTNHYAYEEMSKNISCKILSLSDDLTNAANEIDDLFRTILMLKKPGYLYIPCDLVNVEIDASNLQSVPANKLRERVPSTDSQTIAKITSTIVDKLLSSSNPVVLCDILTDRYGMTAYAQDLVDSLKVPCCNSFMGKALLNESKEHYIGDFNGEESNKMVHSYISNTDCFLHIGDYYNEINSGHWSLYNGINKESIVILNPEYVKIGSQTYQNVSFEDILPAILSSIKANPNLPCFHIPKIMSTIEQIPSNTPISQTLMLEKLQSFLKPNDVLVTETCSLMFGLPDIRMPENSKVIGQHFYLSIGMALPCSFGVSVALNELKKDSRLILIEGDGSAQMTVQELSNFNRENVVKPLIILLNNSGYTVERVIKGPKREYNDIRPDWKWTQLLQTFGMDDAKSMKVTTPEELDDALDEYGNNLSTPRLLEVVLDKLDVPWRFNKMVGN

SEQ ID NO: 11 - protein, 407 aa, Pichia kudriavzevii
MLRLFSRRTPSVRALPKFTRSLATASPEAGAQEVSNLHDIVEIELPEYSFEGYKLDVPELNYSTEKGTLLQMYKDMVIIRRMEMAADALYKAKKIRGFCHLSVGQEAIAVGIENAITKQDDIITSYRCHGTTYMRGASVQEVLAELMGRRSGVSYGKGGSMHMYTKGFYGGNGIVGAQVPLGTGLAFAHHYRDQKNMTWTMYGDGAANQGQVFESFNMAKLWNLPCVFTCENNKYGMGTSASRSSAMTEYYKRGQYIPGLKVNGMDILAVYQAAKFAKEWTSNDNGPLVIEFETYRYGGHSMSDPGTTYRTREEVQNMRSKKDPIAGLKAHLLEFNIATEEEIKAFDKSARKYVDEQVKLADASPPPEAKMSILFEDVYVPGSEIPVLRGRIRDDSWSFEKGGFAYK

SEQ ID NO: 12 - protein, 388 aa, Pichia kudriavzevii
MVSPAERLSTIASTIKPNRKDSTSLQPEDYPEHPFKVTVVGSGNWGCTIAKVIAENTVERPRQFQRDVNMWVYEELIEGEKLTEIINTKHENVKYLPGIKLPVNVVAVPDIVEACAGSDLIVFNIPHQFLPRILSQLKGKVNPKARAISCLKGLDVNPNGCKLLSTVITEELGIYCGALSGANLAPEVAQCKWSETTVAYTIPDDFRGKGKDIDHQILKSLFHRPYFHVRVISDVAGISIAGALKNVVAMAAGFVEGLGWGDNAKAAVMRIGLVETIQFAKTFFDGCHAATFTHESAGVADLITTCAGGRNVRVGRYMAQHSVSATEAEEKLLNGQSCQGIHTTREVYEFLSNMGRTDEFPLFTTTYRIIYENFPIEKLPECLEPVED

SEQ ID NO: 13 - protein, 388 aa, Pichia kudriavzevii; identical to SEQ ID NO: 12

SEQ ID NO: 14 - protein, 374 aa, Pichia kudriavzevii
MAKVNYPEEFAGFAIVDTNEWNKPKYITYKPKTFGDHDIDIEIECCGLCGSDIMTARGGEAGWGQITLPQVVGHEIIGKVIKVGPKVTLHRLGDRVGLGAQAFACLDCNRCKQDNEQYCPHGVTTYDGKYPDGYVSQGGYASHVRAHEHLCFPIPEKLQSIHAASLCCAGLTVFSPLKRYIPKDLPNGVKAKVAIVGLGGLGHLAVQLCKALGAEPWVFSRGNSKKEQAFKLGASGFVATGEKDWEKPLFDQFDLILNCAIGLSGLNLDAFISILKVDGRFCSVGLPSESEKYDVSPFTFFSNGSCLCSSALGSREEALELFELAAENNIVPWVETIPISEKGCEEALTRAWDGDVRYRFVFTEFDKAFGTGTS

SEQ ID NO: 15 - protein, 501 aa, Pichia kudriavzevii
MALPLATTISLSSGKTLEQPIGLFIDNEFVNPISVSNARTLTTFNPSTGEPITDVHCASAADVDVAVNAANKAMETWKDIDPTVRVELLLKLASLVDEHSQAIAEIEALDSGKPLYSNALADVQSVAEYLRYCAGWADKLHGTQIPINSKVMAITKRVPLVVGCIIPWNYPISMASWKFCPALAAGCTIVMKSSEITPLSLLYFANLVKLAGFPKGVFNVVSGFGDDVGSALSNHPKLGKIAFTGSTLTGQKVMADAARSNLKSVSLECGGKSPLIVFEDAELDECVKWASFGVMYNTGQNCTANSRIIVHDKVYDQFIEKFLSQLKEDWKMGDVMNEKTTLGPLVSQQQYERVQSYIDIGVKEGATLIQPLKESTPSNGFYISPTVFTNVKEDMRIVKEEIFGPVVTISKFSTEEEAISKANDTIYGLAAMLFTTNFERANRVADKLEAGSVYINSSNNESTKVPFGGMKMSGIGRELGQEAFNLYTVTKSIYYSYGAKL

SEQ ID NO: 16 - protein, 501 aa, Pichia kudriavzevii
MSALFRTIETPNGKTLEQPLGLFIDNEWVKTNRTFETINPSTGEAICHVYRAGVQEVNDAVEAANRAFRNESWSGLTGSQRGDLLYRMYQVIKRDAESIASIESIDNGKPYAAECLDGDLGEAADVFKYYAGWADKITGELIGSSVLGKNKMCYVEPTPLGAVGGIVPWNFPFTMMAWKIAPALATGCTVVMKSSEVTPLTALWYGKIALEVGLPKGVLNILSGFGSDVGSAMASHPKLAKIAFTGSTATGKKIMEAAGGSNLKKVTLECGGKSPYIVFDDADLELAVEWAYWGIWYNKGEVCTSTSRFLIQEDIYDKFVESFVELTKTRAITADPFDDRCTIGPLVSSSQYEKVKKYVEIGKNEGAKLLTGKFIDGPGYFCEPFIFSECTDDMTIMKEEIFGPVVGITKFSTVKEAIERANATTYGLGAALFSSNITKAHSVAAKLEAGMVWINSNGDSDIHIPFGGSKMSGIGRELGPYALDLFTEKKAVHVNLSLPVK

SEQ ID NO: 17 - protein, 502 aa, Pichia kudriavzevii
MGATTAEISIPNGNKYEQPTGLFINGDFVASSDGKTAEVENPGNGNIVCSVHLASIEDINTAVEAAEDAFFKRWATISGKAKGEYLSKIADLIVKYSDQLADLEAIESGKPKDTNAIFDVLHSADVFRYYAGKAVTAQSGKTIESELSKFTYTVYEPYGVCAAIIAWNFPMSTFAWKVAACLAAGNTMVVKTSELTPLSALFMCKIFQEADLPAGVINVTCGLGSVAGVRLSEHEKVQKISFTGSTGVGKLIQESAAKSNLKYCTLECGGKSPLVIYEDADLEQAVKWAAFGIFFNKGEICTASSRIYVQESVYDKFLTMYKDHVEEAYVQGEQFATGVNVGPTVCKAQQEKILAYIESAKQEGGRIITGGKIPSYTNKNGYYLEPTIIADCNQDMKVVREEIFGPVVTVSKFTSDEEAIKLSNDSEYGLAAYLFTKDLVRSQNYIRKVQSGQVFVNFTFAADFRLPFGGYKMSGNGRELGDEGLSAFQQVKAVHINLTGKL

SEQ ID NO: 18 - protein, 497 aa, Pichia kudriavzevii
MSATSVTFPIINETYQQPTGLFINNEFVSAKSGKTFDVNTPIDESLICKVQQADAEDVEIAVQAASKAYKTWRFTPPNERGRYLNKLADLMDEKRDLLAKIESLDNGKALHCAKFDVNLVIEYFRYCAGYCDKIDGRTITTDVEHFTYTRKEPLGVCGAITPWNFPLLMFAWKIGPALATGNTIILKPASATPLSNLFTCTLIKEAGIPAGVVNVVPGSGRGCGNSILQHPKIKKVAFTGSTEVGKTVMKECANSIKKVTLELGGKSPNIVFKDCNVEQTIQNLITGIFFNGGEVCCAGSRIYIEATDEKWYTEFLTKFKETVEKLKIGNPFEEGVFQGAQTTPDQFQTVLDYITAANESSLKLLTGGKRIGNKGYFVEPTIFYDVPQNSKLTQEEIFGPVAVVLPFKSTEELIEKANDSDFGLGSGIHTEDFNKAIWVSERLEAGSVWINTYNDFHPAAPFGGYKESGIGREMGIEAFDNYTQTKLVRARVNKPAF

SEQ ID NO: 19 - protein, 567 aa, synthetic construct
MSTGVKANDVKTKTKGADLVVDCLIAQGVTHVFGIPGAKIDAVFDVLQDRGPELIVCRHEQNAAFMAAAVGRLTGKPGVCLVTSGPGASNLATGLVTANAEGDPVVALAGAVPRADRLKRTHQSMDNAALFEPITKYSVEVEHPDNIPEALTNAFRAATSGNPGAAFVSLPQDVVTAETTVKSIGALSAPQLGPAPDEAISAVVAKIKSAKLPVILLGMRASRPEVTKAVRKLLAKTELPVVETYQAAGAISRELEDHFFGRVGLFRNQPGDILLEQADLVITIGYDPIEYDPKFWNKLGDRTIIHLDDIQADIDHDYQPELELIGDIALTVNSIAEDLPKLVLSSKSEEVLEDLRAKLSEQAEVPARAKEGLTHPLQVIRTLRSLIDDDTTVTCDIGSHAIWMARYFRSYEPRRLLFSNGMQTLGVALPWAIAATLVNPGEKVVSVSGDGGFLFSAMELETAVRLKAPIVHLVWNDGTYDMVAFQQMMKYGRTSAVEFGDVDIVKYAESFGAKGLRVNSPDELEDVLKAALDAEGPVIIDIPIDYRDNIKLSEKLLPNQFNEMKKA

SEQ ID NO: 20 - protein, 340 aa, synthetic construct
MAVKVYYDKDADLSIIQGKKVAIIGYGSQGHAHALNLRDSGVDVIVGLREGSKSFAKAEEDGFKVFTVAEAAKQADVIMILAPDEIQADIYEEEIEPNLKAGNALFFAHGFNIHFGQIKPPADVDVFMVAPKGPGHLVRRTFVEGGGVPCLIAVYQDASGNAKDLALSYAKGIGGARAGVIETTFKEETETDLFGEQAVLCGGLTALVKAGFETLVEAGYAPEMAYFECLHELKLIVDLMYEGGIANMRYSISDTAEYGDYVSGPRVITAETKEAMKAVLADIQDGTFAKRFIAENKAGRPEFNALRAKEAEHPIEKVGAKLRDMMPWVKKNKDVDAKKN

SEQ ID NO: 21 - protein, 340 aa, Lactococcus lactis
MAVTMYYEDDVEVSALAGKQIAVIGYGSQGHAHAQNLRDSGHNVIIGVRHGKSFDKAKEDGFETFEVGEAVAKADVIMVLAPDELQQSIYEEDIKPNLKAGSALGFAHGFNIHFGYIKVPEDVDVFMVAPKAPGHLVRRTYTEGFGTPALFVSHQNASGHAREIAMDWAKGIGCARVGIIETTFKEETEEDLFGEQAVLCGGLTALVEAGFETLTEAGYAGELAYFEVLHEMKLIVDLMYEGGFTKMRQSISNTAEFGDYVTGPRIITDAVKKNMKLVLADIQSGKFAQDFVDDFKAGRPKLTAYREAAKNLEIEKIGAELRKAMPFTQSGDDDAFKIYQ

SEQ ID NO: 22 - protein, 606 aa, synthetic construct
MPKYRSRTITHGRNMAGARALWRATGMKDEDFGKPIIAVANSFTQFVPGHVHLKDLGQLVKEEIEAAGGVPKEFNTIAVDDGIAMGHDGMLYSLPSREIIADSVEYMVNAHCADAMVCISNCDKITPGMLMAAARLNIPAVFVSGGPMEAGKTKLSDGLTKLDLFEAMGAAADGKISDEDVAQIERSACPTCGSCSGMFTANSMNCLTEALGLSLPGNGSILATHADRKELFREAGRAIVELAKRYYEQDDKSILPRDIATKEAFENAMALDMAMGGSTNTVLHLLAIAQEAEVDFTLDDIDRISRRVPHLCKVAPSGQKYHMEDVHRAGGIPAILGELLRAGLLHGDCLTVTGKTLAENLADWDIPPTQDEAVQEFFRAAPGGIRTQKAFSQSCRWDTLDLDRDEGVIRPLENAFSKDGGLAVLKGNLAPDGCVVKTAGVDESILKFTGPARVFESQDEAVEAILNGKVKAGDVVVIRYEGPKGGPGMQEMLYPTSYLKGKGLGKDCALITDGRFSGGTSGLSIGHVSPEAAEGGPIALVEDGDRITIDIPNRSIDLLVSDEELAARRAAWKPPPRPRYVSGALKAYAKLVTSADKGAVRDLSKL

SEQ ID NO: 23 - protein, 547 aa, synthetic construct
MYTVGDYLLDRLHELGIEEIFGVPGDYNLQFLDQIISRKDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATVEIDRVLSALLKERKPVYINLPVDVAAAKAEKPSLPLKKENSTSNTSEQEILNKIEESLKNAKKPVVIAGHEIISFGLEKTVTQFISKTKLPITTLNFGKSAVDEELPSFLGIYNGKLSEESLKEFVESADFILMLGVKLTDSSTGAFTHHLNENKMISLNIDEGKIFNEVIENFDFESLVSSLSDLSGIEYEGKYIDKKQEDFIPSDALLSQDRLWQAVENLTQSNETIVAEQGTSFFGASNIFLKSKSHFIGQPLWGSIGYTFPAALGSQIADKESRHLLFIGDGSLQLTVQELGLAIREKLNPICFIINNDGYTVEREIHGPNQSYNDIPMWNYSKLPEVFGATEDRVVSKIVRTENEFVSVMKEAQADPNRMYWIELVLAKEDAPKLLKKMGKLFAEQNK

SEQ ID NO: 24 - protein, 499 aa, synthetic construct
MSESQVAVLPNVQQFLDRQHGLYIDGRQVASQSEKRLPVFNPATGQAIASTADASEADVDRAVMSAWRAFVDRRWAGRLPAERERILLRFADLVEQHAEELAQLETLEQGKSIAISRAFEVGCTLNWMRYTAGLTTKIAGKTLDLSIPLPPGARYQAWTRKEPVGVVAGIVPWNFPLMIGMWKVMPALAAGCSIVIKPSETTPLTLLRVAELASEAGIPDGVFNVVTGSGAVCGAALTSHPHVAKVSFTGSTATGKQIARTAADRLTRVTLELGGKNPAIVLKDADPQWVIEGLMMGSFLNQGQVCAASSRIYIEAPLFDTLVAGFEQAVKSLSVGPGMSPTAQINPLVSRAHCDKVAAFLDDAQAQGAELIRGAAGPAGQGYYVSPTLVVNPDAGLRLTREEVFGPVVNLVRVADGEEALRLANDTDYGLTASVWTQDLSQALEYTDRLQAGTVWVNSHTLIDANLPFGGMKQSGTGRDFGPDWLDGWCETKSVCVRY